(19) United States
(12) Patent Application Publication
(10) Pub. No.: US 2026/0301967 A1
(43) Pub. Date: Oct. 1, 2026

(54) DISEASE RISK ESTIMATION DEVICE, DISEASE RISK ESTIMATION SYSTEM, DISEASE RISK ESTIMATION METHOD, AND RECORDING MEDIUM

(71) Applicant: NEC Corporation, Tokyo (JP)

(72) Inventors: Fumiyuki NIHEY, Tokyo (JP); Kentaro NAKAHARA, Tokyo (JP); Hiroshi KAJITANI, Tokyo (JP); Chenhui HUANG, Tokyo (JP); Makoto YASUKAWA, Tokyo (JP); Kenichiro FUKUSHI, Tokyo (JP); Yoshitaka NOZAKI, Tokyo (JP); Kosuke NISHIHARA, Tokyo (JP); John Kenji David CLARK, Tokyo (JP); Kazuya OZAKI, Tokyo (JP)

(73) Assignee: NEC Corporation, Tokyo (JP)

(21) Appl. No.: 19/480,416

(22) PCT Filed: Jun. 22, 2023
(86) PCT No.: PCT/JP2023/023044

§ 371 Date: Oct. 31, 2025

(51) Int. Cl. G16H 50/30; G06Q 30/0207; G06Q 40/08; G16H 40/67; G16H 50/20

(52) U.S. Cl. CPC G16H 50/30; G06Q 30/0207; G06Q 40/0822; G16H 40/67; G16H 50/20

(57) ABSTRACT

A disease risk estimation device comprises an acquisition unit that acquires sensor data measured in response to a foot movement of a target person who is an estimation target of the disease risk, a risk estimation unit that uses the acquired sensor data to estimate the disease risk reflecting the risk of each disease, and an output unit that outputs disease risk information according to the estimated disease risk. The disease risk information supports the target person with decision-making an action for the disease risk.

### TECHNICAL FIELD

The present disclosure relates to a disease risk estimation device, a disease risk estimation system, a disease risk estimation method, and a recording medium.

### BACKGROUND ART

With growing interest in healthcare, services that provide information according to gait have attracted attention. For example, a technique for analyzing a gait using sensor data measured by a sensor mounted on footwear such as shoes has been developed. In the time-series data of the sensor data, a feature associated with a gait event related to a physical condition appears. If the disease risk of the subject can be estimated by the feature associated with the gait event, information relevant to the disease risk can be provided to a specialized institution that handles health insurance and life insurance.

PTL 1 discloses an insurance proposal system that proposes a wide variety of insurance plans based on contents caused by lifestyle in daily life. The system of PTL 1 includes a pedestrian database in which gait information of a plurality of pedestrians and sick/injured history information indicating a past sick/injured state stored in association with the gait information are accumulated as pedestrian information. The system of PTL 1 acquires gait information of a user from a footwear module including a sensor unit that detects movement. The system of PTL 1 refers to pedestrian information and calculates an index value serving as an index of an insurance premium according to the gait information of the user.

### CITATION LIST

### Patent Literature

PTL 1: JP 2020-197948 A

### SUMMARY OF INVENTION

### Technical Problem

In the method of PTL 1, an index value relevant to the gait information of the user is calculated with reference to the pedestrian information accumulated in the database. According to the method of PTL 1, if the past sick/injured associated with the gait information is stored in the database, the index value relevant to the gait information can be calculated. The method of PTL 1 discloses an example in which a gait risk value is weighted according to the degree of injuries or illnesses such as a sprain or a bone fracture. However, in the method of PTL 1, the disease risk reflecting a risk for each disease cannot be estimated.

An object of the present disclosure is to provide a disease risk estimation device, a disease risk estimation system, a disease risk estimation method, and a recording medium capable of estimating a disease risk reflecting a risk for each disease using sensor data measured in accordance with movement of a foot.

### Solution to Problem

A disease risk estimation device according to an aspect of the present disclosure includes an acquisition unit that acquires sensor data measured in accordance with a movement of a foot of a subject who is an estimation target of a disease risk, a risk estimation unit that estimates a disease risk reflecting a risk for each disease using the acquired sensor data, and an output unit that outputs disease risk information relevant to the estimated disease risk.

In a disease risk estimation method according to an aspect of the present disclosure, sensor data measured according to a movement of a foot of a subject who is an estimation target of a disease risk is acquired, a disease risk reflecting a risk for each disease is estimated using the acquired sensor data, and disease risk information relevant to the estimated disease risk is output.

A program according to an aspect of the present disclosure causes a computer to execute a process of acquiring sensor data measured in accordance with a movement of a foot of a subject who is an estimation target of a disease risk, a process of estimating a disease risk reflecting a risk for each disease using the acquired sensor data, and a process of outputting disease risk information relevant to the estimated disease risk.

### Advantageous Effects of Invention

According to the present disclosure, it is possible to provide a disease risk estimation device, a disease risk estimation system, a disease risk estimation method, and a recording medium capable of estimating a disease risk reflecting a risk for each disease using sensor data measured in accordance with movement of a foot.

### BRIEF DESCRIPTION OF DRAWINGS

FIG. 1 is a block diagram illustrating an example of a configuration of a disease risk estimation system in the present disclosure.

FIG. 2 is a block diagram illustrating an example of a configuration of a measurement device included in the disease risk estimation system in the present disclosure.

FIG. 3 is a conceptual diagram illustrating an arrangement example of the measurement device of the disease risk estimation system in the present disclosure.

FIG. 4 is a conceptual diagram illustrating an example of a coordinate system set in the measurement device of the disease risk estimation system in the present disclosure.

FIG. 5 is a conceptual diagram illustrating an example of a human body surface used in the description of the present disclosure.

FIG. 6 is a block diagram illustrating an example of a configuration of a disease risk estimation device included in the disease risk estimation system in the present disclosure.

FIG. 7 is a conceptual diagram illustrating an example of a gait cycle used in the description of the present disclosure.

FIG. 8 is a conceptual diagram illustrating an estimation example of a physical ability score in the disease risk estimation system in the present disclosure.

FIG. 9 is a conceptual diagram illustrating an estimation example of a disease risk score in the disease risk estimation system in the present disclosure.

FIG. 10 is a conceptual diagram illustrating an estimation example of the disease risk score in the disease risk estimation system in the present disclosure.

FIG. 11 is a conceptual diagram illustrating an estimation example of the disease risk score in the disease risk estimation system in the present disclosure.

FIG. 12 is a flowchart illustrating an example of an operation of the disease risk estimation system in the present disclosure.

FIG. 13 is a conceptual diagram for explaining an application example of the disease risk estimation system in the present disclosure.

FIG. 14 is a conceptual diagram for explaining an application example of the disease risk estimation system in the present disclosure.

FIG. 15 is a block diagram illustrating an example of a configuration of the disease risk estimation system in the present disclosure.

FIG. 16 is a block diagram illustrating an example of a configuration of the disease risk estimation device included in the disease risk estimation system in the present disclosure.

FIG. 17 is a graph illustrating an example of time-series data of disease risk scores estimated by the disease risk estimation system in the present disclosure.

FIG. 18 is a graph illustrating an example of time-series data of disease risk scores estimated by the disease risk estimation system in the present disclosure.

FIG. 19 is a graph illustrating an example of time-series data of disease risk scores estimated by the disease risk estimation system in the present disclosure.

FIG. 20 is a flowchart illustrating an example of operation of the disease risk estimation system in the present disclosure.

FIG. 21 is a conceptual diagram for explaining an application example of the disease risk estimation system in the present disclosure.

FIG. 22 is a conceptual diagram for explaining an application example of the disease risk estimation system in the present disclosure.

FIG. 23 is a block diagram illustrating an example of a configuration of the disease risk estimation system in the present disclosure.

FIG. 24 is a block diagram illustrating an example of a configuration of the disease risk estimation device included in the disease risk estimation system in the present disclosure.

FIG. 25 is a flowchart illustrating an example of an operation of the disease risk estimation system in the present disclosure.

FIG. 26 is a conceptual diagram for explaining an application example of the disease risk estimation system in the present disclosure.

FIG. 27 is a conceptual diagram for explaining an application example of the disease risk estimation system in the present disclosure.

FIG. 28 is a conceptual diagram for explaining an application example of the disease risk estimation system in the present disclosure.

FIG. 29 is a conceptual diagram for explaining an application example of the disease risk estimation system in the present disclosure.

FIG. 30 is a conceptual diagram for explaining an application example of the disease risk estimation system in the present disclosure.

FIG. 31 is a conceptual diagram for explaining an application example of the disease risk estimation system in the present disclosure.

FIG. 32 is a block diagram illustrating an example of a configuration of the disease risk estimation device included in the disease risk estimation system in the present disclosure.

FIG. 33 is a flowchart for explaining an example of the operation of the disease risk estimation device included in the disease risk estimation system in the present disclosure.

FIG. 34 is a block diagram illustrating an example of a hardware configuration that executes control and processing in the present disclosure.

### EXAMPLE EMBODIMENT

Hereinafter, modes for carrying out the present disclosure will be described with reference to the drawings. In the present disclosure, the drawings used in description of each example embodiment are associated with one or more example embodiments. Elements included in each drawing may apply to one or more example embodiments. The example embodiments described below may have technical limitations for carrying out the present disclosure, but the scope of the disclosure is not limited to the following. In all the drawings used in the following description of the example embodiments, the same reference signs are given to similar parts unless otherwise specified. In the following example embodiments, repeated description of similar configurations and operations may be omitted. The directions of the arrows in the drawings illustrate an example, and do not limit the directions of data, signals, and the like.

### First Example Embodiment

First, an example of a disease risk estimation system in the present disclosure will be described with reference to the drawings. The disease risk estimation system according to the present example embodiment estimates a disease risk related to a specific disease using sensor data related to movement of a foot according to the gait of a subject (user) who is an estimation target of a disease risk. In the present example embodiment, an example of estimating a disease risk reflecting a risk for each disease will be described.

### (Configuration)

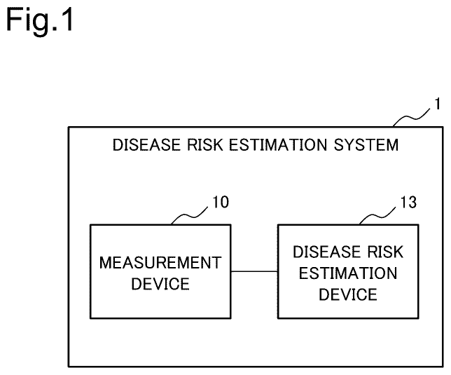

FIG. 1 is a block diagram illustrating an example of a configuration of a disease risk estimation system 1 in the present disclosure. The disease risk estimation system 1 includes a measurement device 10 and a disease risk estimation device 13. For example, the measurement device 10 is installed on footwear of a subject (user) who is an estimation target of the disease risk. For example, the function of the disease risk estimation device 13 is installed in a mobile terminal carried by the subject (user). Hereinafter, configurations of the measurement device 10 and the disease risk estimation device 13 will be individually described.

### [Measurement Device]

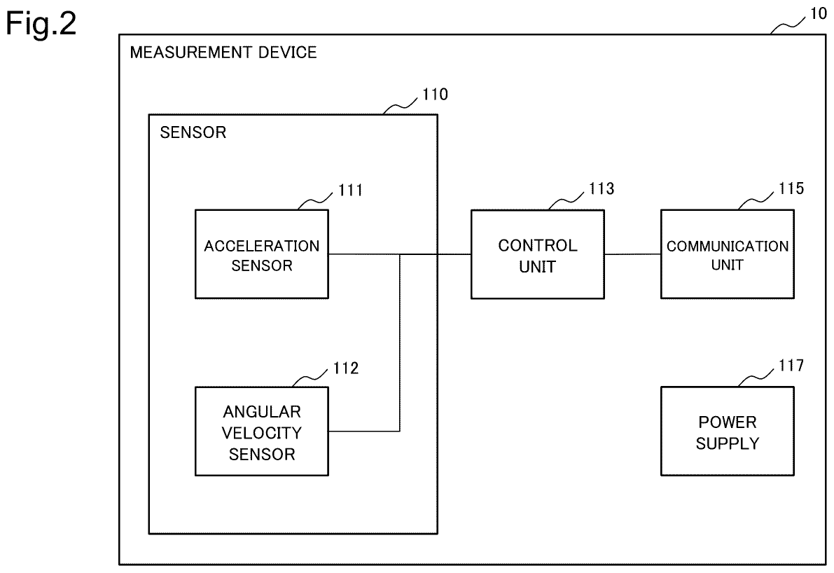

FIG. 2 is a block diagram illustrating an example of a configuration of the measurement device 10. The measurement device 10 includes a sensor 110, a control unit 113, a communication unit 115, and a power supply 117. The sensor 110 includes an acceleration sensor 111 and an angular velocity sensor 112. The sensor 110 may include a sensor other than the acceleration sensor 111 and the angular velocity sensor 112. Sensors other than the acceleration sensor 111 and the angular velocity sensor 112 that can be included in the sensor 110 will not be described.

The acceleration sensor 111 is a sensor that measures accelerations (also referred to as spatial accelerations) in three axial directions. The acceleration sensor 111 measures acceleration (also referred to as spatial acceleration) as a physical quantity related to the movement of the foot. The acceleration sensor 111 outputs the measured acceleration to the control unit 113. For example, a sensor of a piezoelectric type, a piezoresistive type, a capacitance type, or the like can be used as the acceleration sensor 111. The sensor used as the acceleration sensor 111 is not limited as long as it can measure acceleration.

The angular velocity sensor 112 is a sensor that measures angular velocity (also referred to as a spatial angular velocity) around three axes. The angular velocity sensor 112 measures angular velocity (also referred to as a spatial angular velocity) as a physical quantity related to the movement of the foot. The angular velocity sensor 112 outputs the measured angular velocity to the control unit 113. For example, a sensor of a vibration type, a capacitance type, or the like can be used as the angular velocity sensor 112. The sensor used as the angular velocity sensor 112 is not limited as long as the sensor can measure the angular velocity.

The sensor 110 is implemented by, for example, an inertial measurement device that measures acceleration and angular velocity. An example of the inertial measurement device is an inertial measurement unit (IMU). The IMU includes an acceleration sensor 111 that measures acceleration in three axis directions and an angular velocity sensor 112 that measures the angular velocity around the three axes. The sensor 110 may be implemented by an inertial measurement device such as a vertical gyro (VG) or an attitude heading reference system (AHRS). The sensor 110 may be implemented by GPS/INS (Global Positioning System/Inertial Navigation System). The sensor 110 may be implemented by a device other than the inertial measurement device as long as it can measure a physical quantity related to the movement of the foot.

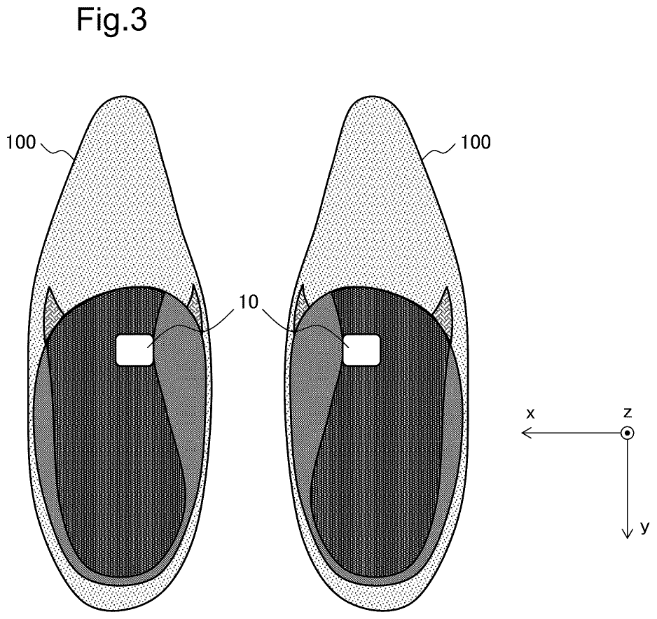

FIG. 3 is a conceptual diagram illustrating an example in which the measurement device 10 is disposed in shoes 100 of both feet. In the example of FIG. 3, the measurement device 10 is installed at a position relevant to the back side of the arch of a foot. For example, the measurement device 10 is disposed in an insole inserted into the shoe 100. For example, the measurement device 10 may be disposed on the bottom surface of the shoe 100. For example, the measurement device 10 may be embedded in the main body of the shoe 100. The measurement device 10 may be detachable from the shoe 100 or may not be detachable from the shoe 100. The measurement device 10 may be installed at a position other than the back side of the arch of the foot as long as the sensor data related to the movement of the foot can be measured. The measurement device 10 may be installed on a sock worn by the user or a decorative article such as an anklet worn by the user. The measurement device 10 may be directly attached to the foot or may be embedded in the foot. As long as data from which the disease risk can be estimated can be measured, the measurement device 10 may be disposed in one shoe 100.

In the example of FIG. 3, a local coordinate system including an x axis in the left-right direction, a y axis in the front-rear direction, and a z axis in the upward-downward direction is set with reference to the measurement device 10 (sensor 110). FIG. 3 illustrates an example in which the same coordinate system is set for the left foot and the right foot. For example, in a case where the sensors 110 produced with the same specifications are disposed in the left and right shoes 100, the upward-downward direction (directions in the Z-axis direction) of the sensors 110 disposed in the left and right shoes 100 are the same. In this case, the three axes of the local coordinate system set in the sensor data derived from the left foot and the three axes of the local coordinate system set in the sensor data derived from the right foot are the same on the left and right. In the present disclosure, the left side of the x axis is positive, the rear side of the y axis is positive, and the upper side of the z axis is positive.

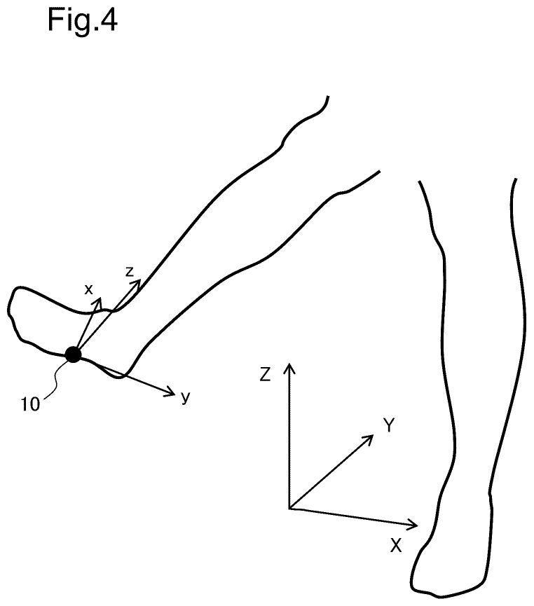

FIG. 4 is a conceptual diagram for explaining a local coordinate system (x axis, y axis, z axis) set in the measurement device 10 (sensor 110) installed on the back side of the arch of the foot and a world coordinate system (X axis, Y axis, Z axis) set with respect to the ground. FIG. 4 illustrates an example in which different coordinate systems are set for the left foot and the right foot. In the world coordinate system (X axis, Y axis, Z axis), the lateral direction of the user is set to the X-axis direction, the direction of the back surface of the user is set to the Y-axis direction, and the gravity direction is set to the Z-axis direction in a state where the user facing the traveling direction is upright. The example of FIG. 4 conceptually illustrates the relationship between the local coordinate system (x axis, y axis, z axis) and the world coordinate system (X axis, Y axis, Z axis), and does not accurately illustrate the relationship between the local coordinate system and the world coordinate system that varies depending on the gait of the user.

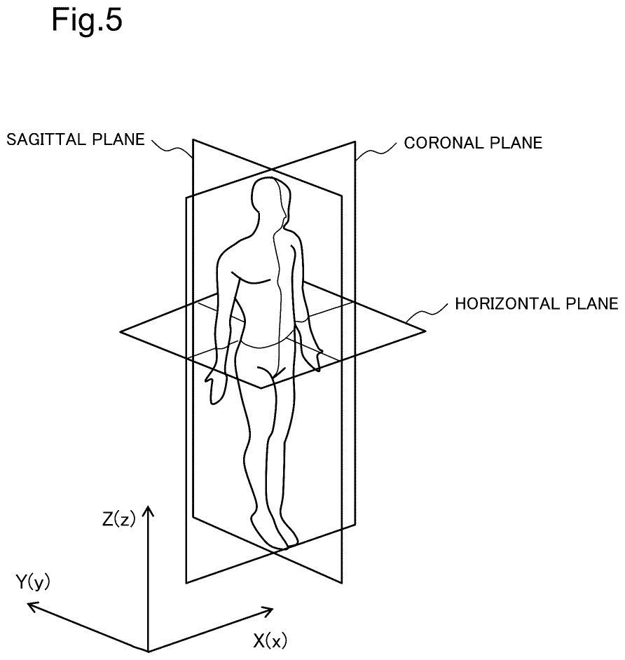

FIG. 5 is a conceptual diagram for explaining a surface (also referred to as a human body surface) set for the human body. In the present example embodiment, a sagittal plane dividing the body into left and right, a coronal plane dividing the body into front and rear, and a horizontal plane dividing the body horizontally are defined. As illustrated in FIG. 5, it is assumed that the world coordinate system and the local coordinate system coincide with each other in a state in which the center line of the foot is oriented in the traveling direction. FIG. 5 illustrates an example in which different coordinate systems are set for the left foot and the right foot. In the present example embodiment, rotation in a sagittal plane with the X axis (x axis) as a rotation axis is defined as a roll, rotation in a coronal plane with the Y axis (y axis) as a rotation axis is defined as a pitch, and rotation in a horizontal plane with the Z axis (z axis) as a rotation axis is defined as a yaw. A rotation angle in the sagittal plane with the X axis (x axis) as a rotation axis is defined as a roll angle, a rotation angle in the coronal plane with the Y axis (y axis) as a rotation axis is defined as a pitch angle, and a rotation angle in a horizontal plane with the Z axis (z axis) as a rotation axis is defined as a yaw angle.

The control unit 113 (control means) causes the acceleration sensor 111 and the angular velocity sensor 112 to measure sensor data. For example, the control unit 113 causes the acceleration sensor 111 and the angular velocity sensor 112 to start measurement in response to a measurement start signal transmitted from the disease risk estimation device 13. For example, the control unit 113 may cause the acceleration sensor 111 and the angular velocity sensor 112 to start measurement in response to detection of the gait of the user. For example, after the heights of both legs/feet in the vertical direction are the same over a predetermined period set in advance, the control unit 113 starts the measurement of the step width from a time point at which movement of one of the right and left feet in the traveling direction is detected as a starting point. The control unit 113 may be configured to start the measurement of the step width at a predetermined timing set in advance.

The control unit 113 acquires accelerations in three axial directions from the acceleration sensor 111. The control unit 113 acquires the angular velocity around three axes from the angular velocity sensor 112. For example, the control unit 113 performs analog-to-digital conversion (AD conversion) on the acquired physical quantities (analog data) such as angular velocity and acceleration. The physical quantities (analog data) measured by the acceleration sensor 111 and the angular velocity sensor 112 may be converted into digital data in each of the acceleration sensor 111 and the angular velocity sensor 112. For example, an AD conversion circuit that performs AD conversion on physical quantities (analog data) such as angular velocity and acceleration may be provided. The control unit 113 outputs the converted digital data (also referred to as sensor data) to the communication unit 115. For example, the control unit 113 may temporarily store the sensor data in a storage unit (not illustrated).

The sensor data includes at least acceleration data converted into digital data and angular velocity data converted into digital data. The acceleration data includes acceleration vectors in three axial directions. The angular velocity data includes angular velocity vectors around three axes. The acceleration data and the angular velocity data are associated with acquisition times of the data. The control unit 113 may add corrections such as mounting error correction, temperature correction, and linearity correction to the acceleration data and the angular velocity data.

For example, the control unit 113 may calculate at least one of gait indexes to be described later. In that case, the measurement device 10 outputs the calculated gait index to the disease risk estimation device 13. For example, the control unit 113 may calculate a feature quantity used for estimation of physical ability described later. In that case, the measurement device 10 outputs the calculated feature quantity to the disease risk estimation device 13.

For example, the control unit 113 is implemented by a microcomputer or a microcontroller that performs overall control and data processing of the measurement device 10. For example, the control unit 113 includes a central processing unit (CPU), a random access memory (RAM), a read only memory (ROM), a flash memory, and the like.

The communication unit 115 (communication means) acquires sensor data from the control unit 113. The communication unit 115 transmits the acquired sensor data to the disease risk estimation device 13. The sensor data transmitted from the communication unit 115 is received by the disease risk estimation device 13. The transmission timing of the sensor data is not particularly limited. For example, the communication unit 115 transmits sensor data at a preset transmission timing. For example, the communication unit 115 transmits the sensor data in real time in response to the measurement of the sensor data. For example, the communication unit 115 may store sensor data measured during a predetermined period and collectively transmit the stored sensor data at a preset timing. For example, the communication unit 115 (communication means) may be configured to receive the measurement start signal from the disease risk estimation device 13. In this case, the communication unit 115 outputs the received measurement start signal to the control unit 113.

For example, the communication unit 115 transmits sensor data to the disease risk estimation device 13 via wireless communication. For example, the communication unit 115 transmits sensor data to the disease risk estimation device 13 via a wireless communication function (not illustrated) conforming to a standard such as Bluetooth (registered trademark) or WiFi (registered trademark). The communication function of the communication unit 115 may conform to a standard other than Bluetooth (registered trademark) or WiFi (registered trademark). The communication unit 115 may transmit the sensor data to the disease risk estimation device 13 via a wire such as a cable.

The power supply 117 is a battery that supplies power for the measurement device 10 to operate. For example, the power supply 117 is implemented by a thin battery such as a coin type or a button type. For example, the power supply 117 is implemented by a primary battery such as a lithium primary battery, a silver oxide battery, an alkaline button battery, or an air zinc battery. In the case of being implemented by the primary battery, the power supply 117 may be implemented by a long-life battery. The power supply 117 may be implemented by a rechargeable secondary battery. In the case of being implemented by the secondary battery, the power supply 117 may be a battery that can be charged in a wired manner or may be a battery that can be charged wirelessly. When the power supply 117 can wirelessly supply power, the wireless power supply device may be disposed at a place where footwear is placed, such as an entrance or a footwear box. If the footwear on which the measurement device 10 is mounted is stacked on the wireless power supply device, the measurement device 10 can be charged appropriately when not in use.

### [Disease Risk Estimation Device]

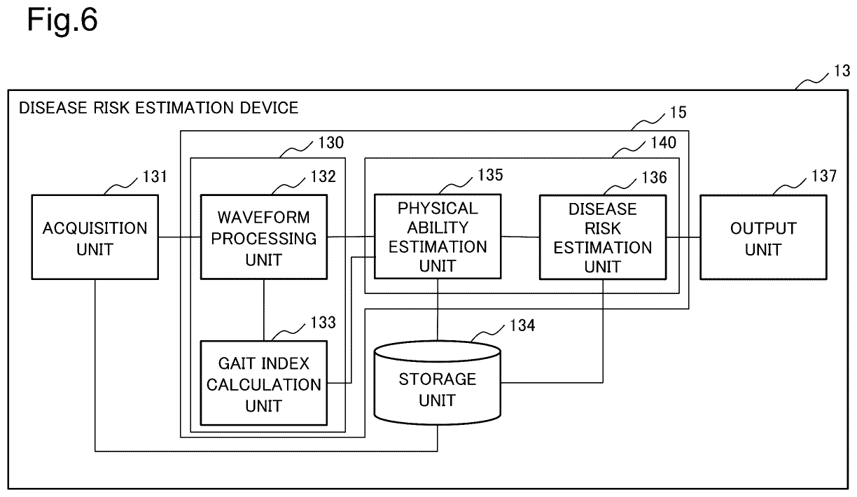

FIG. 6 is a block diagram illustrating an example of a configuration of the disease risk estimation device 13. The disease risk estimation device 13 includes an acquisition unit 131, a waveform processing unit 132, a gait index calculation unit 133, a storage unit 134, a physical ability estimation unit 135, a disease risk estimation unit 136, and an output unit 137. The waveform processing unit 132, the gait index calculation unit 133, the physical ability estimation unit 135, and the disease risk estimation unit 136 constitute a risk estimation unit 15. The waveform processing unit 132 and the gait index calculation unit 133 constitute a calculation unit 130. The physical ability estimation unit 135 and the disease risk estimation unit 136 constitute an estimation unit 140.

The acquisition unit 131 (acquisition means) acquires sensor data from the measurement device 10. The acquisition unit 131 receives sensor data from the measurement device 10 via wireless communication. For example, the acquisition unit 131 receives sensor data from the measurement device 10 via a wireless communication function (not illustrated) conforming to a standard such as Bluetooth (registered trademark) or WiFi (registered trademark). The communication function of the acquisition unit 131 may conform to a standard other than Bluetooth (registered trademark) or WiFi (registered trademark) as long as the communication function can communicate with the measurement device 10. The acquisition unit 131 may receive the sensor data from the measurement device 10 via a wire such as a cable. For example, the acquisition unit 131 may acquire a gait index or a feature quantity calculated by the measurement device 10.

The acquisition unit 131 acquires body information (attribute) of the user. The body information includes gender, date of birth, height, and weight. The date of birth is converted to age. For example, the body information is input via an input device (not illustrated). For example, the body information is input via a mobile terminal used by the user. For example, the body information may be stored in the storage unit 134 in advance. The body information may be updated at an arbitrary timing according to an input by the user.

The waveform processing unit 132 (waveform processing means) acquires sensor data from the acquisition unit 131. The waveform processing unit 132 extracts time-series data for one gait cycle from the time-series data of the acceleration in the three-axis direction and the angular velocity around the three axes included in the sensor data. The time-series data for one gait cycle is also referred to as gait waveform data. The waveform processing unit 132 extracts the gait waveform data based on the timing of the gait event detected from the time-series data of the sensor data. For example, the waveform processing unit 132 extracts the gait waveform data with the timing of the heel strike as a start point and the timing of the next heel strike as an end point.

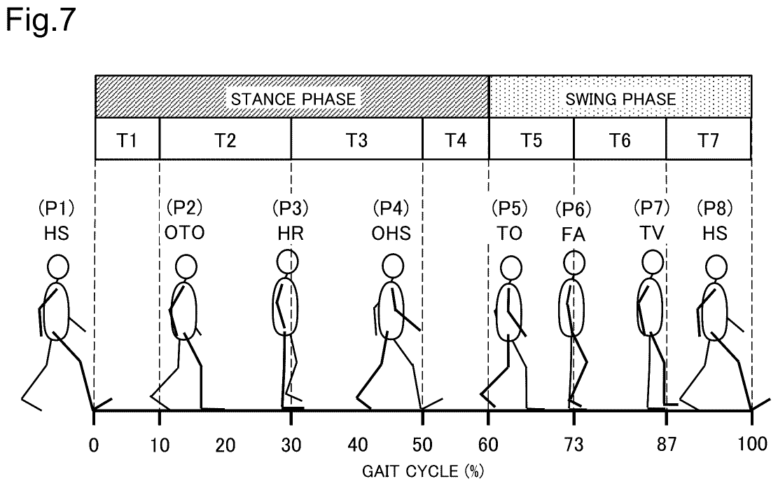

FIG. 7 is a conceptual diagram for explaining one gait cycle with the right foot as a reference. One gait cycle based on the left foot is also similar to that of the right foot. The horizontal axis in FIG. 7 indicates one gait cycle of the right foot with a time point at which the heel of the right foot lands on the ground as a starting point and a time point at which the heel of the right foot next lands on the ground as an ending point. The horizontal axis in FIG. 7 is normalized with one gait cycle as 100%. Normalizing one gait cycle by 100% is referred to as first normalization. The one gait cycle of one foot is roughly divided into a stance phase in which at least a part of the back side of the foot is in contact with the ground and a swing phase in which the back side of the foot is separated from the ground. The stance phase is a period in which at least a part of the back side of the foot is in contact with the ground. The stance phase is further subdivided into an initial stance period T1, a mid-stance period T2, a terminal stance period T3, and a pre-swing period T4. The swing phase is a period in which the back side of the foot is away from the ground. The swing phase is further subdivided into an initial swing period T5, a mid-swing period T6, and a terminal swing period T7. The horizontal axis in FIG. 7 is normalized such that the stance phase is 60% and the swing phase is 40%. The normalization of the gait waveform data so that the stance phase becomes 60% and the swing phase becomes 40% is referred to as second normalization. The period illustrated in FIG. 7 is an example, and the period constituting one gait cycle, the name of those periods, and the like are not limited.

As illustrated in FIG. 7, a plurality of events occur during the gait. In the gait, a plurality of events in the gait are also referred to as gait events. P1 represents an event (heel strike) in which the heel of the right foot touches the ground (HS: Heel Strike). P2 represents an event (opposite toe off) in which the toe of the left foot is separated from the ground in a state where the sole of the right foot is grounded (OTO: Opposite Toe Off). P3 represents an event (heel rise) in which the heel of the right foot lifts in a state where the sole of the right foot is grounded (HR: Heel Rise). P4 is an event (opposite heel strike) in which the heel of the left foot is grounded (OHS: Opposite Heel Strike). P5 represents an event (toe off) in which the toe of the right foot is separated from the ground in a state where the sole of the left foot is grounded (TO: Toe Off). P6 represents an event (foot adjacent) in which the left foot and the right foot cross each other in a state where the sole of the left foot is grounded (FA: Foot Adjacent). P7 represents an event (tibia vertical) in which the tibia of the right foot is approximately perpendicular to the ground in a state where the sole of the left foot is grounded (TV: Tibia Vertical). P8 represents an event (heel strike) in which the heel of the right foot touches the ground (HS: Heel Strike). P8 is relevant to the end point of the gait cycle starting from P1 and is relevant to the start point of the next gait cycle. The gait event illustrated in FIG. 7 is an example, and does not limit the events that occur during the gait or the names of these events.

The timing of the heel strike is the timing of a local minimum peak immediately after a local maximum peak appearing in the time-series data of the acceleration in the traveling direction (acceleration in the Y direction). The local maximum peak serving as a mark of the heel strike timing is relevant to the maximum peak of the gait waveform data for one gait cycle. A section between consecutive heel strikes is relevant to one gait cycle. The timing of the toe off is the rising timing of the local maximum peak appearing after the period of the stance phase in which the fluctuation does not appear in the time-series data of the acceleration in the traveling direction (acceleration in the Y direction). The timing at the midpoint between the timing at which the roll angle is minimum and the timing at which the roll angle is maximum is relevant to the mid-stance period.

The waveform processing unit 132 normalizes the time of the extracted gait waveform data for one gait cycle to a gait cycle of 0 to 100% (percent) (first normalization). Timing such as 1% or 10% included in the 0 to 100% gait cycle is also referred to as a gait phase. The waveform processing unit 132 normalizes the gait waveform data subjected to the first normalization for one gait cycle so that the stance phase becomes 60% and the swing phase becomes 40% (second normalization). By performing the second normalization on the gait waveform data, it is possible to reduce the shift of the gait phase from which the feature quantity is extracted. The waveform processing unit 132 outputs the normalized gait waveform data to the gait index calculation unit 133.

For example, the waveform processing unit 132 extracts/normalizes the gait waveform data for one gait cycle using the acceleration in the traveling direction (acceleration in the Y direction). With respect to acceleration/angular velocity other than the acceleration in the traveling direction (acceleration in the Y direction), the waveform processing unit 132 extracts/normalizes gait waveform data for one gait cycle in accordance with the gait cycle of the acceleration in the traveling direction (acceleration in the Y direction). The waveform processing unit 132 may generate time-series data of the angle around three axes by integrating time-series data of the angular velocity around the three axes. In that case, the waveform processing unit 132 also extracts/normalizes the gait waveform data for one gait cycle in accordance with the gait cycle of the acceleration in the traveling direction (acceleration in the Y direction) with respect to the angle around the three axes.

The waveform processing unit 132 may extract/normalize the gait waveform data for one gait cycle using acceleration/angular velocity other than the acceleration in the traveling direction (acceleration in the Y direction). For example, the waveform processing unit 132 may detect the heel strike and the toe off from the time-series data of the vertical acceleration (acceleration in the Z direction) (omitted in the drawing). The timing of the heel strike is a timing of a steep local minimum peak appearing in the time-series data of the vertical acceleration (acceleration in the Z direction). At the timing of the steep local minimum peak, the value of the vertical acceleration (acceleration in the Z direction) becomes substantially zero. The local minimum peak serving as a mark of the timing of the heel strike is relevant to the minimum peak of the gait waveform data for one gait cycle. A section between consecutive heel strikes is one gait cycle. The timing of the toe off point is a timing of an inflection point in the middle of gradually increasing after the time-series data of the acceleration in the vertical direction (acceleration in the Z direction) passes through a section with a small fluctuation after the local maximum peak immediately after the heel strike. The waveform processing unit 132 may extract/normalize the gait waveform data for one gait cycle using both the acceleration in the traveling direction (acceleration in the Y direction) and the acceleration in the vertical direction (acceleration in the Z direction). The waveform processing unit 132 may extract/normalize the gait waveform data for one gait cycle using acceleration, angular velocity, angle, and the like other than the acceleration in the traveling direction (acceleration in the Y direction) and the acceleration in the vertical direction (acceleration in the Z direction).

The waveform processing unit 132 extracts a feature quantity (physical ability feature quantity) used to estimate the physical ability from the gait waveform data. The waveform processing unit 132 extracts a physical ability feature quantity used to estimate at least one physical ability. For example, the waveform processing unit 132 extracts a physical ability feature quantity used for estimation of at least one of physical abilities such as grip strength (total muscle strength of the whole body), dynamic balance, lower limb muscle strength, movement ability, and static balance. For example, the waveform processing unit 132 extracts the physical ability feature quantity for each gait phase cluster according to a preset condition. The gait phase cluster is a cluster in which temporally continuous gait phases are integrated. The gait phase cluster includes at least one gait phase. The gait phase cluster also includes a single gait phase. The waveform processing unit 132 outputs the extracted physical ability feature quantity to the physical ability estimation unit 135.

The gait index calculation unit 133 (gait index calculation means) acquires the normalized gait waveform data from the waveform processing unit 132. The gait index calculation unit 133 calculates a gait index used for estimating the physical ability using the normalized gait waveform data. The gait index to be calculated is not particularly limited as long as the gait index can be calculated using the normalized gait waveform data. For example, the gait index calculation unit 133 calculates a gait index related to a distance, a height, an angle, a speed, a time, a frailty level, a center of pressure exclusion index (CPEI), and the like. Hereinafter, representative gait indexes will be described. A specific calculation method of the following gait index will be omitted.

For example, the gait index calculation unit 133 calculates an index related to a distance and a height as a gait index. For example, the gait index calculation unit 133 calculates a stride length, an outward turning distance, foot raising height, a foot clearance (FTC), and a minimum toe clearance (MTC). The stride length indicates a distance between a front foot and a rear foot during the gait. The outward turning distance indicates the maximum value of the distance at which the foot is separated outward with respect to the traveling direction in the swing phase. The foot raising height indicates the maximum value of the distance between the measurement device 10 (sensor 110) and the ground in the swing phase. The FTC indicates the maximum value of the distance between the heel and the ground in the swing phase. The MTC indicates the minimum value of the distance between the toe and the ground in the swing phase.

For example, the gait index calculation unit 133 calculates an index related to an angle as the gait index. For example, the gait index calculation unit 133 calculates the grounding angle, the ground separation angle, the toe direction, the roll angle of the heel strike, the roll angle of the toe off, the swing peak angular velocity, and the hallux angle. The grounding angle indicates a maximum value of an angle formed by the sole surface and the ground at the time of heel strike. The ground separation angle indicates an angle formed between the sole surface and the ground in the swing phase. The toe direction indicates an average value of the toe directions with respect to the traveling direction in the swing phase. The roll angle of the heel strike is an angle formed between the ankle and the ground at the time of the heel strike when viewed from the rear viewing seat. The roll angle of the toe off ground is an angle formed between the ankle and the ground at the time of kicking as viewed from the rear viewing seat. The swing peak angular velocity is the angular velocity in the ankle joint dorsiflexion direction in a section from immediately after kicking until the toe comes into closest contact with the ground. The hallux angle indicates an angle at which the thumb of the foot is inclined toward the index finger. Specifically, the hallux angle is an angle formed by the center line of the first metatarsal and the center line of the first proximal phalanx.

For example, the gait index calculation unit 133 calculates an index related to a speed as the gait index. For example, the gait index calculation unit 133 calculates a gait speed, cadence, and a maximum speed in swing. The gait speed indicates a speed in the gait. The cadence indicates the number of steps per minute. The maximum speed in swing indicates a speed at which the user swings out the leg in the swing phase.

For example, the gait index calculation unit 133 calculates an index related to a time as the gait index. For example, the gait index calculation unit 133 calculates a standing time, a load time, a plantar grounding time, a kicking time, a swing time, and a double support time (DST). The standing time indicates a time during which the foot is grounded during the gait. The standing time is a sum of the load time, the plantar grounding time, and the kicking time. The load time is a time from when the heel is grounded to when the toe is grounded in the stance phase. The plantar grounding time is a time during which the entire plantar surface is grounded and the plantar surface and the ground are horizontal in the stance phase. The kicking time is a time until the toe kicks the ground from the state of the sole grounding in the stance phase. The swing time indicates a time during which the foot is separated from the ground during the gait. The DST is divided into DST1 and DST2. DST1 indicates a time during which the foot on which the measurement device 10 (sensor 110) is mounted is in front of the opposite foot in a period in which both feet are simultaneously grounded. DST2 indicates a time during which the foot on which the measurement device 10 (sensor 110) is mounted is behind the opposite foot in a period in which both feet are simultaneously grounded.

For example, the gait index calculation unit 133 calculates a frailty level or a center of pressure exclusion index (CPEI) as the gait index. The frailty level is an estimated value of the frail state according to the gait state. For example, the gait index calculation unit 133 estimates indexes such as a determination result R₁ indicating health, a determination result R₂ indicating a possibility of frailty, and a determination result R₃ having a high possibility of frailty as the frailty level. The CPEI indicates an estimated value of the expansion ratio of the movement of the center of foot pressure applied to the ground during the stance phase.

The storage unit 134 (storage means) stores a physical ability estimation model (described later) for estimating the physical ability using the physical ability feature quantity extracted from the gait waveform data. For example, the physical ability is at least one of grip strength, dynamic balance, lower limb muscle strength, movement ability, and static balance. The physical ability may include other than grip strength, dynamic balance, lower limb muscle strength, movement ability, and static balance. The storage unit 134 stores a physical ability estimation model learned for a plurality of subjects. For example, the physical ability estimation model outputs an index (physical ability score) related to the physical ability according to the input of the physical ability feature quantity extracted from the gait waveform data.

The storage unit 134 stores a disease risk estimation model (described later) for estimating a disease risk using the body information, the gait index, and the physical ability score. The disease risk indicates a risk of acquiring a specific disease. For example, the specific diseases include gout, diabetes, hypertension, nephrolithiasis, liver cirrhosis, arteriosclerosis, thromboembolism, dyslipidemia, hypercholesterolemia, and hyperlipidemia. For example, the specific diseases include low back pain, sleep apnea syndrome, insomnia, depression, knee osteoarthritis, and Parkinson's syndrome. The specific disease may include diseases other than those described above. The storage unit 134 stores a disease risk estimation model learned for a plurality of subjects. For example, the disease risk estimation model outputs an index related to the disease risk (disease risk score) according to the input of the body information, the gait index, and the physical ability score.

For example, the physical ability estimation model and the disease risk estimation model may be stored in the storage unit 134 at the time of factory shipment of a product. The physical ability estimation model and the disease risk estimation model may be stored in the storage unit 134 at a timing such as at the time of calibration before the user uses the disease risk estimation device 13. For example, a physical ability estimation model and a disease risk estimation model stored in a storage device (not illustrated) such as an external server may be used. In that case, it is sufficient that the physical ability estimation model and the disease risk estimation model can be accessed via an interface (not illustrated) connected to the storage device.

The storage unit 134 stores body information (attribute) of the user. The body information includes gender, date of birth, height, and weight. The date of birth is converted to age. The body information may be updated at any timing.

The physical ability estimation unit 135 (physical ability estimation means) acquires the physical ability feature quantity extracted from the gait waveform data from the waveform processing unit 132. The physical ability estimation unit 135 acquires the body information (attribute) stored in the storage unit 134. The physical ability estimation unit 135 estimates the physical ability score using the physical ability feature quantity and the body information (attribute). The physical ability estimation unit 135 inputs the physical ability feature quantity and the user's body information (attribute) to the physical ability estimation model stored in the storage unit 134. For example, the physical ability estimation unit 135 estimates a physical ability score related to the physical ability of at least one of grip strength (total muscle strength of the whole body), dynamic balance, lower limb muscle strength, movement ability, or static balance. The estimation of the physical ability score by the physical ability estimation unit 135 will be described later. The physical ability estimation unit 135 outputs the physical ability score output from the physical ability estimation model to the disease risk estimation unit 136.

For example, the physical ability estimation unit 135 may estimate the body information (attribute) using the gait index calculated by the gait index calculation unit 133. For example, the physical ability estimation unit 135 estimates the body information (attribute) using a gait index correlated with the body information (attribute). For example, when muscle strength decreases due to aging, a decrease in gait speed, cadence, and the like is observed. Therefore, if a gait speed, cadence, or the like is used, an age group can be estimated even if an accurate age cannot be estimated. For example, there is also a correlation between height and stride length. Therefore, if the stride length is used, even if an accurate age cannot be estimated, an age group can be estimated. By combining specific gait indexes, it is also possible to estimate the age more accurately. With such a configuration, the body information can be estimated using the sensor data measured by the measurement device 10 without inputting any of the body information such as the height, the weight, the age, and the gender. It is also assumed that there is a user who does not want to input information such as age, weight, body mass index (BMI), and shoe size. In order to estimate such a disease risk of the user, it is useful to estimate the body information (attribute) using the gait index. For example, the physical ability estimation unit 135 may compare an input value of the body information (attribute) with an estimated value. When the deviation between the input value and the estimated value of the body information (attribute) is large, there is a possibility that there is an error in the input value input by the user. In such a case, a notification or a warning prompting confirmation of the input value of the body information (attribute) may be notified to the terminal device or the like of the user.

For example, the estimated value of the body information (attribute) is stored in the storage unit 134. The body information (attribute) may be estimated by any of the waveform processing unit 132, the gait index calculation unit 133, the physical ability estimation unit 135, and the disease risk estimation unit 136 included in the risk estimation unit 15. For example, a component that estimates body information (attribute) may be added to the disease risk estimation device 13. For example, the acquisition unit 131 may acquire an estimated value of body information (attribute) estimated by an external estimation device (not illustrated).

Next, an estimation example of the physical ability score by the physical ability estimation unit 135 will be described with an example. Here, an example of feature quantities used for estimation of grip strength (total muscle strength of the whole body), dynamic balance, lower limb muscle strength, movement ability, and static balance will be described. The following examples do not limit the physical ability estimated by the physical ability estimation unit 135. The physical ability estimated by the physical ability estimation unit 135 may be appropriately selected according to a disease for which a disease risk is to be estimated.

### <Grip Strength (Total Muscle Strength of Whole Body)>

There is a correlation between grip strength, which is one of physical abilities, and total muscle strength of the whole body. The grip strength is also correlated with the knee extension strength. For example, the estimated value of the grip strength is an index of the total muscle strength. For example, a score relevant to the estimated value of the grip strength (also referred to as a total muscle strength score) is an index of the total muscle strength. The total muscle strength score is a value obtained by scoring the grip strength, which is an index of the total muscle strength, on a preset basis. The grip strength is affected by attributes such as gender, age, and height. Therefore, the total muscle strength score may be scored based on a criterion for each attribute. In particular, the grip strength is affected by gender. Therefore, the total muscle strength score may be scored based on different criteria depending on the gender. The index of the total muscle strength is not limited to the grip strength as long as the total muscle strength can be scored.

The gait phase in which the feature quantity used to estimate the grip strength is extracted differs depending on the gender. For male, there is a correlation between the activities of quadriceps muscles and the grip strength. Therefore, in the estimation of the grip strength of the male, the feature quantity extracted from the gait phase in which the feature of the activities of quadriceps muscles appears is used. For female, there is a correlation between the activities of the vastus lateralis, the vastus intermedius, and the vastus medialis of the quadriceps muscles and the grip strength. Therefore, in the estimation of the grip strength of a female, the feature quantity extracted from the gait phase in which the features of the activities of the vastus lateralis, the vastus intermedius, and the vastus medialis appear is used.

A feature quantity AM1, a feature quantity AM2, a feature quantity AM3, and a feature quantity AM4 are used to estimate the grip strength of a male. The feature quantity AM1 is extracted from the section of the gait phase 3% of the gait waveform data related to the time-series data of the acceleration in the traveling direction (acceleration in the Y direction). The gait phase 3% is included in the initial stance period T1. The feature quantity AM1 mainly includes features related to the movements of the vastus lateralis, the vastus intermedius, and the vastus medialis of the quadriceps muscles. The feature quantity AM2 is extracted from the section of the gait phase 59 to 62% of the gait waveform data related to the time-series data of the acceleration in the traveling direction (acceleration in the Y direction). The gait phase 59 to 62% is included in the pre-swing period T4. The feature quantity AM2 mainly includes a feature related to movement of the rectus femoris muscle of the quadriceps muscles. The feature quantity AM3 is extracted from the section of the gait phase 59 to 62% of the gait waveform data related to the time-series data of the vertical acceleration (acceleration in the Z direction). The gait phase 59 to 62% is included in the pre-swing period T4. The feature quantity AM3 mainly includes a feature related to movement of the rectus femoris muscle of the quadriceps muscles. The feature quantity AM4 is a ratio (DST1) of a period from the heel strike to the opposite toe off in the period in which both feet are simultaneously grounded. DST1 is a ratio of a period from the heel strike to the opposite toe off in one gait cycle. The feature quantity AM4 mainly includes a feature caused by the quadriceps muscles.

A feature quantity AF1, a feature quantity AF2, and a feature quantity AF3 are used to estimate the grip strength of a female. The feature quantity AF1 is extracted from a section of the gait phase 13% of the gait waveform data related to the time-series data of the acceleration in the lateral direction (acceleration in the X direction). The gait phase 13% is included in the mid-stance period T2. The feature quantity AF1 mainly includes features related to the movements of the vastus lateralis, the vastus intermedius, and the vastus medialis of the quadriceps muscles. The feature quantity AF2 is extracted from a section of the gait phase 7 to 10% of the gait waveform data related to the time-series data of the angular velocity (pitch angular velocity) in the coronal plane (around the Y axis). The gait phase 7 to 10% is included in the initial stance period T1. The feature quantity AF2 mainly includes features related to movements of the vastus lateralis, the vastus intermedius, and the vastus medialis. The feature quantity AF3 is a ratio (DST2) of a period from the opposite heel strike to the toe off in the period in which both feet are simultaneously grounded. DST2 is a ratio of a period from the opposite heel strike to the toe off in one gait cycle. The sum of DST1 and DST2 is relevant to a period in which both feet are simultaneously grounded in one gait cycle. The feature quantity AF3 mainly includes features related to movements of the vastus lateralis, the vastus intermedius, and the vastus medialis.

### <Dynamic Balance>

Dynamic balance, one of physical abilities, can be evaluated by the performance of a functional reach test (FRT). In the present disclosure, the performance of the FRT is evaluated by the distance between the fingertips (also referred to as functional reach distance) in a state where both hands are raised 90 degrees with respect to the horizontal plane and the upper limb is moved forward as much as possible. The functional reach distance (hereinafter, referred to as an FR distance) is a performance value of the FRT. The larger the FR distance, the higher the FRT performance. Dynamic balance may be assessed outside of the FRT performed with both hands. For example, dynamic balance may be assessed in terms of performance on one-handed FRT or other variations of the FRT.

An index of the dynamic balance is an FR distance. For example, an estimated value of the FR distance is an index of the dynamic balance. For example, a score relevant to the estimated value of the FR distance (also referred to as a dynamic balance score) is an index of the dynamic balance. The dynamic balance score is a value obtained by scoring an FR distance, which is an index of the dynamic balance, based on a preset criterion. The dynamic balance is affected by attributes such as height. Therefore, the dynamic balance score may be scored based on a criterion for each attribute. The index of the dynamic balance is not limited to the FR distance as long as the dynamic balance can be scored. The FR distance is correlated with the activities of the gluteus medius, the iliacus muscle, the hamstrings (the long head of the biceps femoris), the tibialis anterior, and the like, and the magnitude of the compensatory motion that makes the orientation of the toe outward. Therefore, feature quantities extracted from the gait phase in which these features appear are used to estimate the FR distance.

A feature quantity B1, a feature quantity B2, a feature quantity B3, a feature quantity B4, and a feature quantity B5 are used to estimate the FR distance. The feature quantity B1 is extracted from the section of the gait phase 75 to 79% of the gait waveform data related to the time-series data of the acceleration in the traveling direction (acceleration in the Y direction). The gait phase 75 to 79% is included in the mid-swing period T6. The feature quantity B1 mainly includes a feature related to movement of the tibialis anterior and the short head of the biceps femoris. The feature quantity B2 is extracted from the section of the gait phase 62% of the gait waveform data related to the time-series data of the vertical acceleration (acceleration in the Z direction). The gait phase 62% is included in the initial swing period T5. The feature quantity B2 mainly includes a feature related to movement of the iliacus muscle. The feature quantity B3 is extracted from the section of the gait phase 7 to 8% of the gait waveform data related to the time-series data of the angular velocity in the coronal plane (around the Y axis). The gait phase 7 to 8% is included in the initial stance period T1. The feature quantity B3 mainly includes a feature related to the movement of the gluteus medius. The feature quantity B4 is extracted from the section of the gait phase 57 to 58% of the gait waveform data related to the time-series data of the angle (posture angle) in the horizontal plane (around the Z axis). The gait phase 57 to 58% is included in the pre-swing period T4. The feature quantity B4 mainly includes a feature related to a compensatory motion. The compensatory motion is a motion of changing the foot angle to acquire stability in order to compensate for a decrease in balance ability and muscle function associated with aging. The feature quantity B5 is an average value of the foot angles in the horizontal plane in the swing phase. For example, the feature quantity B5 is an average value in the swing phase of the gait waveform data. In other words, the feature quantity B5 is an integral value of the gait waveform data related to the time-series data of the angular velocity in the horizontal plane (around the Z axis). The feature quantity B5 mainly includes a feature related to a compensatory motion.

### <Lower Limb Muscle Strength>

Lower limb muscle strength, which is one of physical abilities, can be evaluated by the performance of a chair standing test. In the present disclosure, the performance of a five-time chair standing test in which standing and sitting from a chair is repeated five times is evaluated. The five-time chair standing test is also referred to as an SS-5 (Sit to Stand-5) test. The performance of the five-time chair standing test is evaluated by the time for repeating standing-up and sitting-down from a chair five times (also referred to as a sit and stand time). The sit and stand time is a performance value of the SS-5 test. The shorter the sit and stand time, the higher the performance in the SS-5 test. The performance may be evaluated by a score of a 30-second chair standing (CS-30) test for measuring the number of times of sit and stand motions from a chair in 30 seconds.

The index of lower limb muscle strength is a sit and stand time. For example, an estimated value of time taking for sitting and standing five times is an index of lower limb muscle strength. For example, a score relevant to the estimated value of the sit and stand time (also referred to as lower limb muscle strength score) is an index of lower limb muscle strength. The lower limb muscle strength score is a value obtained by scoring a sit and stand time, which is an index of lower limb muscle strength, based on a preset criterion. Lower limb muscle strength is affected by attributes such as age. Therefore, the lower limb muscle strength score may be scored based on a criterion for each attribute. The index of lower limb muscle strength is not limited to the sit and stand time as long as lower limb muscle strength can be scored. The sit and stand time is correlated with quadriceps muscles, hamstrings, tibialis anterior, and gastrocnemius muscle. Therefore, feature quantities extracted from the gait phase in which these features appear are used to estimate the sit and stand time.

The estimation of lower limb muscle strength includes a feature quantity C1, a feature quantity C2, a feature quantity C3, and a feature quantity C4. The feature quantity C1 is extracted from the section of the gait phase 42 to 54% of the gait waveform data related to the time-series data of the angular velocity in the sagittal plane (around the X axis). The gait phase 42 to 54% is a section from the terminal stance period T3 to the pre-swing period T4. The feature quantity C1 mainly includes a feature related to movement of the gastrocnemius muscle. The feature quantity C2 is extracted from the section of the gait phase 99 to 100% of the gait waveform data related to the time-series data of the angular velocity in the coronal plane (around the Y axis). The gait phase 99 to 100% is the end stage of the terminal swing period T7. The feature quantity C2 mainly includes features related to movement of the quadriceps muscles, hamstrings, and tibialis anterior. The feature quantity C3 is extracted from the section of the gait phase 10 to 12% of the gait waveform data related to the time-series data of the angular velocity in the coronal plane (around the Y axis). The gait phase 10 to 12% is the early stage of the mid-stance period T2. The feature quantity C3 mainly includes features related to movement of the quadriceps muscles, hamstrings, and gastrocnemius muscles. The feature quantity C4 is extracted from the section of the gait phase 99% of the gait waveform data related to the time-series data of the angle (posture angle) in the horizontal plane (around the Z axis). The gait phase 99% is the end stage of the terminal swing period T7. The feature quantity C4 mainly includes features related to movement of the quadriceps muscles, hamstrings, and tibialis anterior.

### <Movement Ability>

Movement ability, one of physical abilities, can be assessed by TUG (Time Up and Go) test performance. In the present disclosure, the performance of the TUG test is evaluated by the time (also referred to as TUG required time) from standing up from a chair, walking to a mark 3 m (meters) ahead to change the direction, and sitting down again on the chair. The TUG required time is a performance value of the TUG test. The shorter the TUG required time, the higher the TUG test performance. Movement ability may be assessed by test performance on movement ability other than the TUG test.

The index of the movement ability is the TUG required time. For example, the estimated value of the TUG required time is an index of the movement ability. For example, a score relevant to the estimated value of the TUG required time (also referred to as a movement ability score) is an index of the movement ability. The movement ability score is a value obtained by scoring the TUG required time, which is an index of the movement ability, based on a preset criterion. Movement ability is affected by attributes such as age. Therefore, the movement ability score may be scored based on a criterion for each attribute. The index of the movement ability is not limited to the TUG required time as long as the movement ability can be scored. The TUG required time is correlated with the quadriceps muscles, the gluteus medius, and the tibialis anterior muscle. Therefore, feature quantities extracted from the gait phase in which these features appear are used to estimate the TUG required time.

A feature quantity D1, a feature quantity D2, a feature quantity D3, a feature quantity D4, a feature quantity D5, and a feature quantity D6 are used to estimate the movement ability. The feature quantity D1 is extracted from a section of the gait phase 64 to 65% of the gait waveform data related to the time-series data of the acceleration in the lateral direction (acceleration in the X direction). The gait phase 64 to 65% is included in the initial swing period T5. The feature quantity D1 mainly includes a feature related to the motion of the quadriceps muscles in the sit and stand motion. The feature quantity D2 is extracted from the section of the gait phase 57 to 58% of the gait waveform data related to the time-series data of the angular velocity in the sagittal plane (around the X axis). The gait phase 57 to 58% is included in the pre-swing period T4. The feature quantity D2 mainly includes a feature related to the motion of the quadriceps muscles related to the leg kicking speed. The feature quantity D3 is extracted from a section of the gait phase 19 to 20% of the gait waveform data related to the time-series data of the angular velocity in the coronal plane (around the Y axis). The gait phase 19 to 20% is included in the mid-stance period T2. The feature quantity D3 mainly includes a feature related to the movement of the gluteus medius in the direction change. The feature quantity D4 is extracted from the section of the gait phase 12 to 13% of the gait waveform data related to the time-series data of the angular velocity in the horizontal plane (around the Z axis). The gait phase 12 to 13% is the early stage of the mid-stance period T2. The feature quantity D4 mainly includes a feature related to the movement of the gluteus medius in the direction change. The feature quantity D5 is extracted from the section of the gait phase 74 to 75% of the gait waveform data related to the time-series data of the angular velocity in the horizontal plane (around the Z axis). The gait phase 74 to 75% is the early stage of the mid-swing period T6. The feature quantity D5 mainly includes a feature related to the movement of the tibialis anterior in standing and sitting and direction change. The feature quantity D6 is extracted from the section of the gait phase 76 to 80% of the gait waveform data related to the time-series data of the angle (posture angle) in the coronal plane (around the Y axis). The gait phase 76 to 80% is included in the mid-swing period T6. The feature quantity D6 mainly includes a feature related to the movement of the tibialis anterior in standing and sitting and direction change.

### <Static Balance>

Static balance, one of physical abilities, can be assessed by the performance of a single-leg standing test. In the present disclosure, the performance of the single-leg standing test is evaluated for a time (also referred to as a single-leg standing time) during which the eyes are closed and one leg is kept raised from the ground by 5 cm (centimeter). The single-leg standing time is a performance value of static balance. The larger the single-leg standing time, the higher the static balance performance. Static balance may be assessed with a performance other than the closed-eyes single-leg standing test. For example, the static balance may be evaluated in a single-leg standing test with eyes open (open-eyes single-leg standing test) or other variations of the single-leg standing test.

The static balance index is the single-leg standing time. For example, the estimated value of the single-leg standing time is an index of the static balance. For example, a score relevant to the estimated value of the single-leg standing time (also referred to as a static balance score) is an index of the static balance. The static balance score is a value obtained by scoring a single-leg standing time which is an index of the static balance based on a preset criterion. The static balance is affected by attributes such as age and height. Therefore, the static balance score may be scored based on a criterion for each attribute. The index of the static balance is not limited to the single-leg standing time as long as the static balance can be scored. The single-leg standing time is correlated with the gluteus medius, the adductor longus, the sartorius, and the adductor muscles. Therefore, the feature quantity extracted from the gait phase in which these features appear is used to estimate the single-leg standing time.

For the estimation of the static balance, a feature quantity E1, a feature quantity E2, a feature quantity E3, a feature quantity E4, a feature quantity E5, a feature quantity E6, and a feature quantity E7 are used. The feature quantity E1 is extracted from a section of the gait phase 13 to 19% of the gait waveform data related to the time-series data of the acceleration in the lateral direction (acceleration in the X direction). The gait phase 13 to 19% is included in the mid-stance period T2. The feature quantity E1 mainly includes a feature related to the movement of the gluteus medius. The feature quantity E2 is extracted from the section of the gait phase 95% of the gait waveform data related to the time-series data of the vertical acceleration (acceleration in the Z direction). The gait phase 95% is the end stage of the terminal swing period T7. The feature quantity E2 mainly includes a feature related to the movement of the gluteus medius. The feature quantity E3 is extracted from a section of the gait phase 64 to 65% of the gait waveform data related to the time-series data of the angular velocity in the coronal plane (around the Y axis). The gait phase 64 to 65% is included in the initial swing period T5. The feature quantity E3 mainly includes a feature related to movement of the adductor longus and the sartorius. The feature quantity E4 is extracted from the section of the gait phase 11 to 16% of the gait waveform data related to the time-series data of the angular velocity in the horizontal plane (around the Z axis). The gait phase 11 to 16% is included in the mid-stance period T2. The feature quantity E4 mainly includes a feature related to the movement of the gluteus medius. The feature quantity E5 is extracted from the section of the gait phase 57 to 58% of the gait waveform data related to the time-series data of the angular velocity in the horizontal plane (around the Z axis). The gait phase 57 to 58% is included in the pre-swing period T4. The feature quantity E5 mainly includes a feature related to movement of the adductor longus and the sartorius. The feature quantity E6 is extracted from the section of the gait phase 100% of the gait waveform data related to the time-series data of the angle (posture angle) in the horizontal plane (around the Z axis). The gait phase 100% is relevant to the timing of heel strike that is switched from the terminal swing period T7 to the initial stance period T1. The feature quantity of the gait waveform data in the gait phase 100% is relevant to a foot angle in a state where the sole is grounded. The feature quantity E6 mainly includes a feature related to the movement of the gluteus medius. The feature quantity E7 is a distance (rotating amount) between the traveling axis and the foot at a timing when the central axis of the foot is farthest from the traveling axis in the swing phase. The feature quantity E7 is a rotating amount normalized by the height of the subject. The feature quantity E7 mainly includes a feature related to the movement of the abductor and adductor muscles.

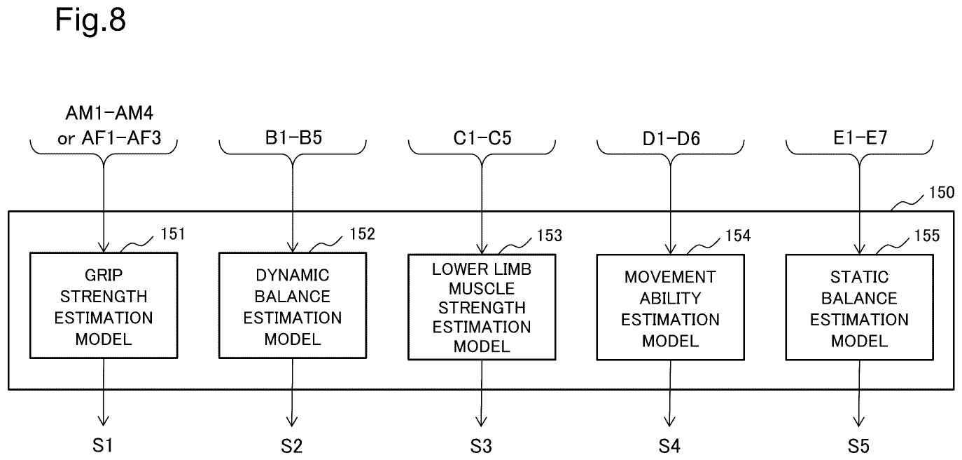

FIG. 8 is a conceptual diagram illustrating an example of a physical ability estimation model 150 for estimating physical ability. The feature quantity extracted from the gait waveform data is input to the physical ability estimation model 150 that estimates physical ability. In addition to the feature quantity data extracted from the gait waveform data, the user's body information (attribute) is input. In FIG. 8, the body information (attribute) input to the physical ability estimation model 150 is omitted. According to the input of the physical ability feature quantity extracted from the gait waveform data, the physical ability estimation model 150 outputs the physical ability score related to the physical ability. In the example of FIG. 8, the physical ability estimation model 150 includes a grip strength estimation model 151, a dynamic balance estimation model 152, a lower limb muscle strength estimation model 153, a movement ability estimation model 154, and a static balance estimation model 155. Each of the grip strength estimation model 151, the dynamic balance estimation model 152, the lower limb muscle strength estimation model 153, the movement ability estimation model 154, and the static balance estimation model 155 outputs a score for each estimation target of the model. The physical ability estimation model 150 may be configured by a single model instead of a model for each physical ability. The physical ability estimation model 150 may be a physical ability value such as a grip strength, an FR distance, a sit and stand time, a TUG required time, and a single-leg standing time, instead of the physical ability score.

The grip strength estimation model 151 outputs a grip strength score S1 related to the grip strength (total muscle strength of the whole body) according to the inputs of the feature quantities AM1 to AM4 or the feature quantities AF1 to AF3. For example, the grip strength estimation model 151 may be a model that outputs the grip strength according to the inputs of the feature quantities AM1 to AM4 or the feature quantities AF1 to AF3. For example, the grip strength estimation model 151 may be different models for men and women. The estimation result of the grip strength estimation model 151 is not limited as long as the estimation result related to the index of the grip strength is output according to the input of the physical ability feature quantity for estimating the total muscle strength. For example, the grip strength estimation model 151 may be a model that outputs the grip strength according to the inputs of the feature quantities AM1 to AM4 or the feature quantities AF1 to AF3. For example, the grip strength estimation model 151 may be a model that estimates the grip strength using attribute data such as age and height in addition to the feature quantities AM1 to AM4 or the feature quantities AF1 to AF3.

The dynamic balance estimation model 152 outputs a dynamic balance score S2 related to the dynamic balance according to the inputs of the feature quantities B1 to B5. The estimation result of the dynamic balance estimation model 152 is not limited as long as the estimation result related to the index of the dynamic balance is output according to the input of the physical ability feature quantity for estimating the dynamic balance. For example, the dynamic balance estimation model 152 may be a model that outputs the FR distance according to inputs of the feature quantities B1 to B5. For example, the dynamic balance estimation model 152 may be a model that estimates the dynamic balance using attribute data such as height in addition to the feature quantities B1 to B5.

The lower limb muscle strength estimation model 153 outputs a lower limb muscle strength score S3 related to lower limb muscle strength according to the inputs of the feature quantities C1 to C4. The estimation result of the lower limb muscle strength estimation model 153 is not limited as long as the estimation result related to the index of the lower limb muscle strength is output according to the input of the physical ability feature quantity for estimating the lower limb muscle strength. For example, the lower limb muscle strength estimation model 153 may be a model that outputs the lower limb muscle strength score S3 related to the lower limb muscle strength according to the inputs of the feature quantities C1 to C4. For example, the lower limb muscle strength estimation model 153 may be a model that estimates the dynamic balance using attribute data such as age in addition to the feature quantities C1 to C4.

The movement ability estimation model 154 outputs a movement ability score S4 related to the movement ability according to the inputs of the feature quantities D1 to D6. The estimation result of the movement ability estimation model 154 is not limited as long as the estimation result related to the index of the movement ability is output according to the input of the physical ability feature quantity for estimating the movement ability. For example, the movement ability estimation model 154 may be a model that outputs the TUG required time according to the inputs of the feature quantities D1 to D6. For example, the movement ability estimation model 154 may be a model that estimates the movement ability using attribute data such as age in addition to the feature quantities D1 to D6.

The static balance estimation model 155 outputs a static balance score S5 related to the static balance according to the inputs of the feature quantities E1 to E7. The estimation result of the static balance estimation model 155 is not limited as long as the estimation result related to the index of the static balance is output according to the input of the physical ability feature quantity for estimating the static balance. For example, the static balance estimation model 155 may be a model that outputs the single-leg standing time according to inputs of the feature quantities E1 to E7. For example, the static balance estimation model 155 may be a model that estimates the static balance using attribute data such as age and height in addition to the feature quantities E1 to E7.

The physical ability estimation model 150 may be stored in an external storage device constructed in a cloud, a server, or the like. In that case, the physical ability estimation unit 135 uses the physical ability estimation model 150 via an interface (not illustrated) connected to the storage device. The physical ability estimation model 150 is a machine learning model. For example, the physical ability estimation model 150 is a model in which a data set having body information (attributes) and gait indexes related to a plurality of subjects as explanatory variables and a score related to physical ability as an objective variable is learned as training data. The physical ability estimation model 150 may be a model in which a data set having body information (attributes) and gait waveform data related to a plurality of subjects as explanatory variables and a score related to the physical ability as an objective variable is learned as training data. For example, the physical ability estimation model 150 may be a model obtained by learning training data in which gait waveform data of acceleration in three axis directions, angular velocity around three axes, and angle (posture angle) around three axes are included in explanatory variables.

For example, the physical ability estimation model 150 may be generated by learning using a linear regression algorithm. For example, the physical ability estimation model 150 may be generated by learning using an algorithm of a support vector machine (SVM). For example, the physical ability estimation model 150 may be generated by learning using a Gaussian process regression (GPR) algorithm. For example, the physical ability estimation model 150 may be generated by learning using a random forest (RF) algorithm. For example, the physical ability estimation model 150 may be generated by unsupervised learning that classifies the subject from whom the physical ability feature quantity is generated according to the input of the physical ability feature quantity. The algorithm for learning the physical ability estimation model 150 is not particularly limited.

The disease risk estimation unit 136 (disease risk estimation) acquires an estimation result (physical ability score) of the physical ability estimated by the physical ability estimation unit 135. The disease risk estimation unit 136 acquires the gait index from the gait index calculation unit 133. Furthermore, the disease risk estimation unit 136 acquires the body information (attribute) of the user from the storage unit 134. The disease risk estimation unit 136 estimates the disease risk reflecting a risk for each disease using the physical ability score, the gait index, and the body information (attribute). For example, the disease risk estimation unit 136 may be configured to estimate the disease risk reflecting a risk for each disease using at least the gait index. For example, the disease risk estimation unit 136 generates disease risk information including advice according to a disease risk generated by being applied to a preset document format. For example, the disease risk information may be generated using a large-scale language model.

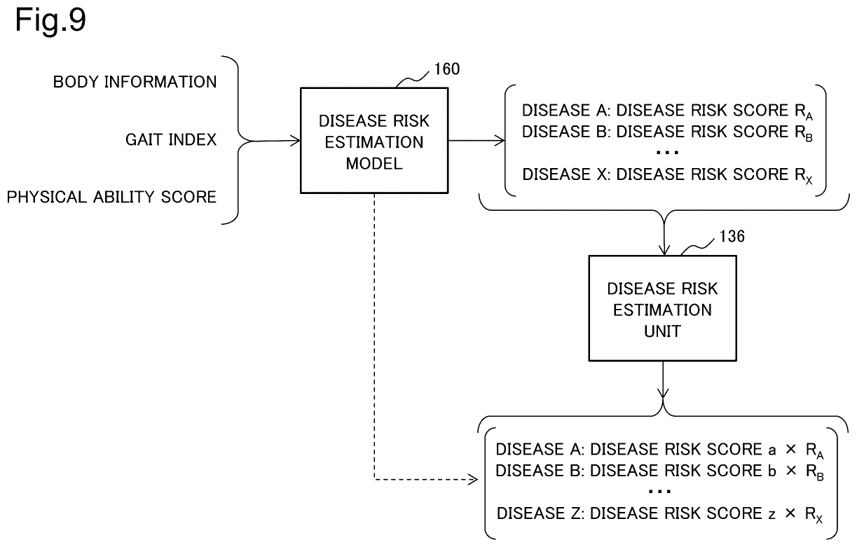

FIG. 9 is a conceptual diagram illustrating an example of disease risk estimation by the disease risk estimation unit 136. The disease risk estimation unit 136 inputs body information, a gait index, and a physical ability score used for estimating a disease risk related to a specific disease to a disease risk estimation model 160. The body information, the gait index, and the physical ability score used for estimating a disease risk related to a specific disease are input to the disease risk estimation model 160. According to the input of the body information, the gait index, and the physical ability score, the disease risk estimation model 160 outputs the disease risk score related to the specific disease. In the example of FIG. 9, the disease risk score is estimated for each of a plurality of diseases. The disease risk estimation model 160 may be configured by a model for each disease or may be configured by a single model. As the number of pieces of data used for estimation increases, the estimation accuracy of the disease risk score by the disease risk estimation model 160 improves.

For example, the disease risk estimation model 160 outputs a disease risk score related to a specific disease such as a lifestyle-related disease. For example, the disease risk estimation model 160 outputs disease risk scores related to specific diseases such as gout, diabetes, hypertension, nephrolithiasis, liver cirrhosis, arteriosclerosis, thromboembolism, dyslipidemia, hypercholesterolemia, and hyperlipidemia. For example, the disease risk estimation model 160 includes low back pain, sleep apnea syndrome, insomnia, depression, knee osteoarthritis, Parkinson's syndrome, and the like. The disease risk estimation model 160 may be configured to output a disease risk score related to a disease other than the above. For example, the disease risk estimation model 160 may be configured to estimate the disease risk score including the test item data of the medical examination.

The disease risk estimation model 160 may be stored in an external storage device constructed in a cloud, a server, or the like. In that case, the disease risk estimation unit 136 uses the disease risk estimation model 160 via an interface (not illustrated) connected to the storage device. The disease risk estimation model 160 is a machine learning model. For example, the disease risk estimation model 160 is a model obtained by learning, as training data, a data set having body information (attribute), a gait index, and physical ability which are related to a plurality of subjects as explanatory variables and having a disease risk score related to a specific disease as an objective variable. For example, the disease risk estimation model 160 may be a model learned using training data in which gait waveform data of acceleration in three axis directions, angular velocity around three axes, and angle (posture angle) around three axes are included in explanatory variables.

For example, the disease risk estimation model 160 is generated by learning using a linear regression algorithm. For example, the disease risk estimation model 160 is generated by learning using an algorithm of a support vector machine (SVM). For example, the disease risk estimation model 160 is generated by learning using a Gaussian process regression (GPR) algorithm. For example, the disease risk estimation model 160 is generated by learning using a random forest (RF) algorithm. For example, the disease risk estimation model 160 may be generated by unsupervised learning that classifies a subject from whom the feature quantity data is generated according to the feature quantity data. The algorithm for learning the disease risk estimation model 160 is not particularly limited.

For example, the disease risk estimation model 160 may be a machine learning model such as an incomplete heterogeneous variational autoencoder or a random forest. In the case of the incomplete heterogeneous variational autoencoder, the disease risk of the subject (user) can be estimated even if there are some defects in features such as the body information (attribute), the gait index, and the body information.

In the example of FIG. 9, the disease risk estimation unit 136 calculates the disease risk score reflecting a risk for each disease by multiplying the disease risk score output from the disease risk estimation model 160 by the weight for each disease. The weight for each disease is a value reflecting the risk of the disease. For example, the weight for each disease is set to a value relevant to the rank indicating the risk of the disease. For example, the weight for a disease having a higher rank is set to a larger value than the weight for a disease having a lower rank. In the example of FIG. 9, the weight a, the weight b, . . . , and the weight z are set for each of the disease A, the disease B, . . . , and the disease Z. For example, in a case where the disease A has a higher risk than the disease B, the weight a is set to a larger value than the weight b. In the example of FIG. 9, the disease risk score of the disease A is a×R_{A}, the disease risk score of the disease B is b×R_{B}, and the disease risk score of the disease Z is z×R_{Z}. The disease risk estimation model 160 may be configured to output the disease risk score reflecting a risk for each disease according to the input of the body information, the gait index, and the physical ability score.

The weight for each disease is set according to an index in a case of suffering from a disease. For example, the weight for each disease is set according to an index such as a death rate, a life expectancy, and a medical cost in a case of suffering from a disease. For example, the higher the death rate, the larger the weight is set. For example, the shorter the life expectancy, the larger the weight is set. For example, the larger the medical cost, the larger the weight is set. The index in the case of suffering from a disease is not limited to the above, and may be a value relevant to the risk of a disease.

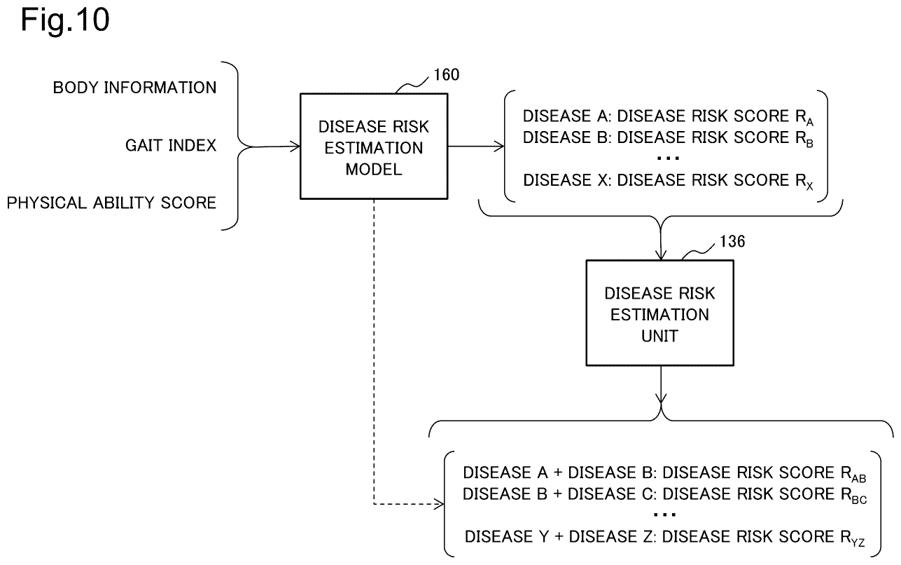

FIG. 10 is a conceptual diagram illustrating an example of disease risk estimation by the disease risk estimation unit 136. In the example of FIG. 10, the disease risk estimation unit 136 calculates a disease risk score relevant to the risk of a combination of diseases. For example, a combination of diabetes and heart disease is at high risk. Therefore, the disease risk score becomes a large value for a combination thereof.

In the example of FIG. 10, the disease risk estimation unit 136 inputs body information, a gait index, and a physical ability score used for estimating a disease risk related to a specific disease to a disease risk estimation model 160. For example, the disease risk estimation unit 136 calculates the disease risk score reflecting a risk for each combination of diseases by multiplying the disease risk score output from the disease risk estimation model 160 by the weight for each combination of diseases. The weight for each combination of diseases is a value reflecting the risk according to the combination of diseases. For example, the weight for each combination of diseases is set to a value relevant to the rank indicating the risk of the combination of diseases to be estimated. For example, the weight for a combination of diseases having a higher rank is set to a larger value than the weight for a combination of diseases having a lower rank. For example, the disease risk estimation unit 136 calculates the product of the disease risk scores reflecting the combined risk for each disease as the disease risk score for each combination of diseases. The disease risk estimation unit 136 may calculate the disease risk score for a combination of three or more diseases. In the example of FIG. 10, the disease risk score related to the combination of the disease A and the disease B is R_{AB}, and the disease risk score related to the combination of the disease B and the disease C is R_{BC}. The disease risk score related to the combination of the disease Y and the disease Z is R_{YZ}. The disease risk estimation model 160 may be configured to output a disease risk score reflecting a risk for each combination of diseases according to the inputs of the body information, the gait index, and the physical ability score.

The weight for each combination of diseases is set according to an index in a case of suffering from the diseases. For example, the weight for each combination of diseases is set according to indexes such as a death rate, a life expectancy, and a medical cost in a case of suffering from the diseases. For example, the higher the death rate, the larger the weight for each combination of diseases is set. For example, the shorter the life expectancy, the larger the weight for each combination of diseases is set. For example, the larger the medical cost, the larger the weight for each combination of diseases is set. The index in the case of suffering from two or more diseases is not limited to the above, and may be a value relevant to the risk of the disease.

For example, the disease risk estimation model may be a model that outputs the annual average receipt issuance number according to inputs of the body information, the gait index, and the physical ability score. The annual average receipt issuance number is relevant to the number of times an individual visits a hospital per year in examination of a specific disease. In that case, the disease risk estimation unit 136 calculates the disease risk score using the annual average receipt issuance number. For example, the disease risk estimation model is generated by learning with a data set in which body information (attribute), a gait index, and physical ability which are related to a plurality of subjects are used as explanatory variables and the annual average number of receipts related to a specific disease is used as an objective variable as training data.

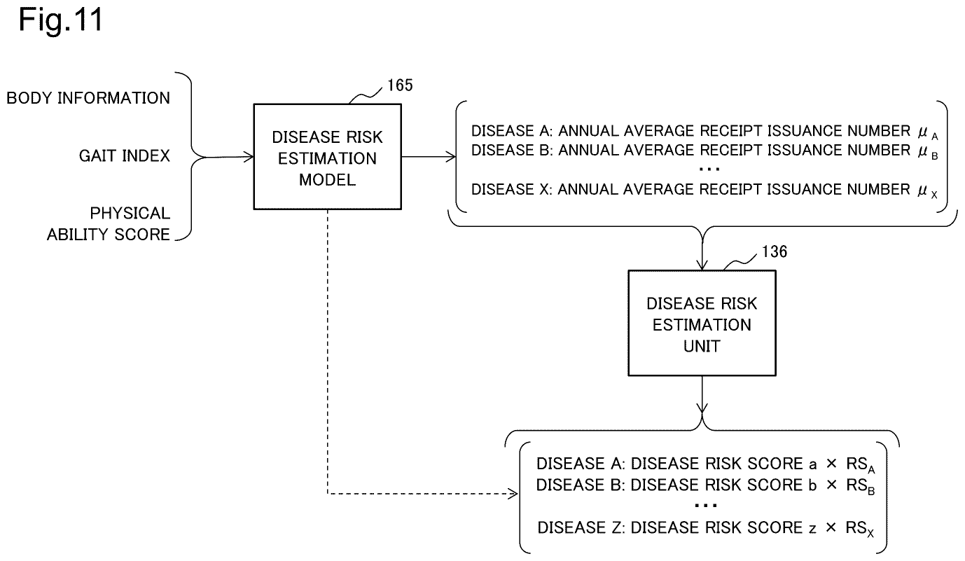

FIG. 11 is a conceptual diagram illustrating an example of a disease risk estimation model 165 that estimates the annual average receipt issuance number. The disease risk estimation unit 136 inputs the body information, the gait index, and the physical ability score to the disease risk estimation model 165. The body information, the gait index, and the physical ability score used for estimating a disease risk related to a specific disease are input to the disease risk estimation model 165. According to the input of the body information, the gait index, and the physical ability score, the disease risk estimation model 165 outputs the annual average receipt issuance number related to the specific disease. In the example of FIG. 11, the annual average receipt issuance number is estimated for each of a plurality of diseases. The disease risk estimation unit 136 calculates a disease risk score using the annual average receipt issuance number output from the disease risk estimation model 165.

Here, an example in which the disease risk estimation unit 136 calculates the disease risk score using the annual average receipt issuance number will be described. Hereinafter, three calculation examples will be described. It is assumed that an annual average receipt issuance number μ₀ of a standard person is obtained in advance. The disease risk estimation model 165 outputs an annual average receipt issuance number μ related to the specific disease according to the input of the body information, the gait index, and the physical ability score related to the estimation subject of the disease risk.

In the first method, the disease risk estimation unit 136 calculates, as the disease risk score, a ratio between the annual average receipt issuance number μ₀ of a standard person and the annual average receipt issuance number μ estimated for the user. The disease risk estimation unit 136 calculates a disease risk score RS₁ using the following Expression 1.

$$\begin{matrix}[ Math . 1 ] & \\ R {S}_{1} = \frac{{μ}_{0}}{μ} & ( 1 )\end{matrix}$$

In the second method, the disease risk estimation unit 136 calculates the disease risk score on the assumption that the annual average receipt issuance number for a specific disease follows the Poisson distribution. In the second method, the disease risk estimation unit 136 calculates a ratio between a probability mass function P₀(X=k) of the annual average receipt issuance number of a standard person and a probability mass function P(X=k) of the annual average receipt issuance number estimated for the user as a disease risk score (k is a natural number). The disease risk estimation unit 136 calculates a disease risk score RS₂ using the following Expression 2.

$$\begin{matrix}[ Math . 2 ] & \\ R {S}_{2} = \frac{P ( X > 0 )}{{P}_{0} ( X > 0 )} = \frac{1 - exp ( - μ )}{1 - exp ( - {μ}_{0} )} & ( 2 )\end{matrix}$$

In the third method, the disease risk estimation unit 136 calculates an odds ratio of the annual average receipt issuance number for a specific disease. The disease risk estimation unit 136 calculates a disease risk score RS₃ using the following Expression 3.

$$\begin{matrix}[ Math . 3 ] & \\ R {S}_{3} = \frac{[ ( > 0 ) / \{ 1 - ( > 0 ) \} ]}{[ {}_{0} ( > 0 ) / \{ 1 - {}_{0} ( > 0 ) \} ]} = \frac{exp ( ) - 1}{exp ( {}_{0} ) - 1} & ( 3 )\end{matrix}$$

The above three calculation examples are merely examples, and do not limit the method of calculating the disease risk score using the annual average receipt issuance number. The disease risk estimation unit 136 may be configured to calculate the disease risk score using an index other than the annual average receipt issuance number.

In the example of FIG. 11, the disease risk estimation unit 136 calculates the disease risk score reflecting a risk for each disease by multiplying the disease risk score RS for each disease calculated using the annual average receipt issuance number by the weight for each disease. Similarly to the example of FIG. 9, in the example of FIG. 11, a weight set to a value relevant to the rank indicating the risk of the disease to be estimated is used. In the example of FIG. 11, the disease risk score of the disease A is a×RS_{A}, the disease risk score of the disease B is b×RS_{B}, and the disease risk score of the disease Z is z×RS_{Z}. The disease risk estimation model 160 may be configured to output the score reflecting a risk for each disease according to the input of the body information, the gait index, and the physical ability score. The disease risk estimation unit 136 may be configured to output a score reflecting a risk for each combination of diseases.

The output unit 137 (output means) outputs disease risk information relevant to the disease risk score estimated by the disease risk estimation unit 136. For example, the output unit 137 displays the disease risk information on the screen of the mobile terminal of the subject (user). For example, the output unit 137 outputs the disease risk information to an external system or the like that uses the disease risk information. The use of the output disease risk information is not particularly limited. For example, the disease risk information is used for statistical analysis, disease prevention research, and the like.

For example, the disease risk estimation device 13 is connected to an external system or the like built in a cloud or a server via a mobile terminal (not illustrated) carried by a subject (user). The mobile terminal (not illustrated) is a portable communication device. For example, the mobile terminal is a portable communication device having a communication function, such as a smartphone, a smart watch, or a mobile phone. For example, the disease risk estimation device 13 is connected to a mobile terminal via wireless communication. For example, the disease risk estimation device 13 is connected to a mobile terminal via a wireless communication function (not illustrated) conforming to a standard such as Bluetooth (registered trademark) or WiFi (registered trademark). The communication function of the disease risk estimation device 13 may conform to a standard other than Bluetooth (registered trademark) or WiFi (registered trademark). For example, the disease risk estimation device 13 may be connected to a mobile terminal via a wire such as a cable. The disease risk information may be used by an application installed in the mobile terminal. In that case, the mobile terminal executes processing using the disease risk information by application software or the like installed in the mobile terminal.

### (Operation)

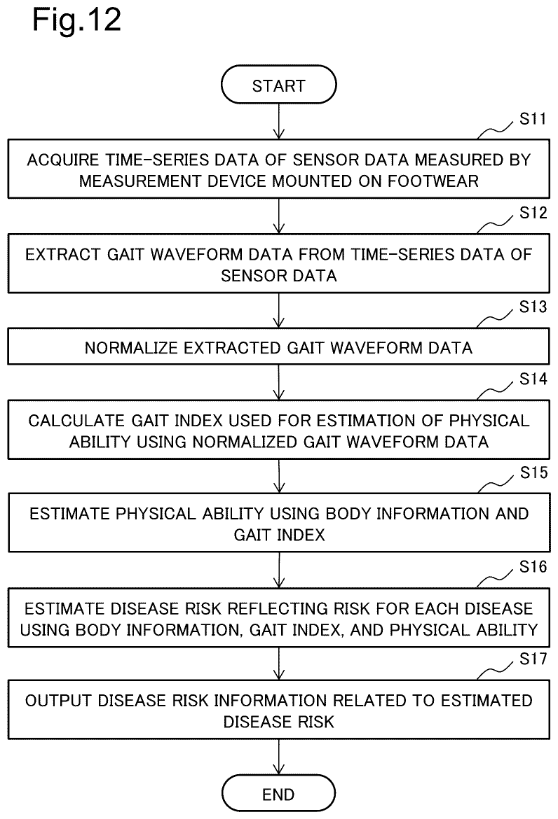

Next, the operation of the disease risk estimation system 1 will be described with reference to the drawings. Hereinafter, the operation of the disease risk estimation device 13 included in the disease risk estimation system 1 will be described. FIG. 12 is a flowchart for explaining an example of the operation of the disease risk estimation device 13. In the description of the processing along the flowchart of FIG. 12, the components of the disease risk estimation device 13 will be described as the operation subject. The operation subject of the processing along the flowchart of FIG. 12 may be the disease risk estimation device 13.

In FIG. 12, first, the acquisition unit 131 acquires time-series data of sensor data measured by the measurement device 10 mounted on the footwear (step S11). The sensor data includes acceleration in three axis directions and angular velocity around three axes.

Next, the waveform processing unit 132 extracts the gait waveform data from the time-series data of the sensor data (step S12). The gait waveform data is relevant to time-series data of sensor data for one gait cycle.

Next, the waveform processing unit 132 normalizes the extracted gait waveform data (step S13). The waveform processing unit 132 performs the first normalization on the gait waveform data in one gait cycle of 100%. The waveform processing unit 132 performs the second normalization on the gait waveform data so that the stance phase becomes 60% and the swing phase becomes 40%.

Next, the gait index calculation unit 133 calculates a gait index used for estimating the physical ability using the normalized gait waveform data (step S14). For example, the gait index calculation unit 133 calculates a gait index related to a distance, a height, an angle, a speed, a time, a frailty level, CPEI, and the like.

Next, the physical ability estimation unit 135 estimates the physical ability using the body information and the gait index (step S15). For example, the physical ability estimation unit 135 estimates physical ability scores such as grip strength (total muscle strength of the whole body), dynamic balance, lower limb muscle strength, movement ability, and static balance.

Next, the disease risk estimation unit 136 estimates the disease risk reflecting a risk for each disease using the body information, the gait index, and the physical ability (step S16). The disease risk estimation unit 136 estimates a disease risk score reflecting a risk for each disease. For example, the disease risk estimation unit 136 estimates a disease risk score reflecting a risk for each disease such as gout, diabetes, hypertension, nephrolithiasis, liver cirrhosis, arteriosclerosis, thromboembolism, dyslipidemia, hypercholesterolemia, or hyperlipidemia. For example, the disease risk estimation unit 136 estimates a disease risk score reflecting a risk for each disease such as low back pain, sleep apnea syndrome, insomnia, depression, knee osteoarthritis, and Parkinson's syndrome.

Next, the output unit 137 outputs disease risk information related to the estimated disease risk (step S17). For example, the output unit 137 displays the disease risk information on the screen of the mobile terminal of the subject (user). For example, the output unit 137 outputs the disease risk information to an external system or the like that uses the disease risk information.

### Application Example

Next, an application example according to the present example embodiment will be described with reference to the drawings. In the following application example, an example of estimating the disease risk using the feature quantity data measured by the measurement device 10 arranged in the shoe will be described. For example, the function of the disease risk estimation device 13 is installed in a mobile terminal carried by a user. The function of the disease risk estimation device 13 may be implemented in a server or a cloud connected to a mobile terminal carried by the user in a data-communicable manner.

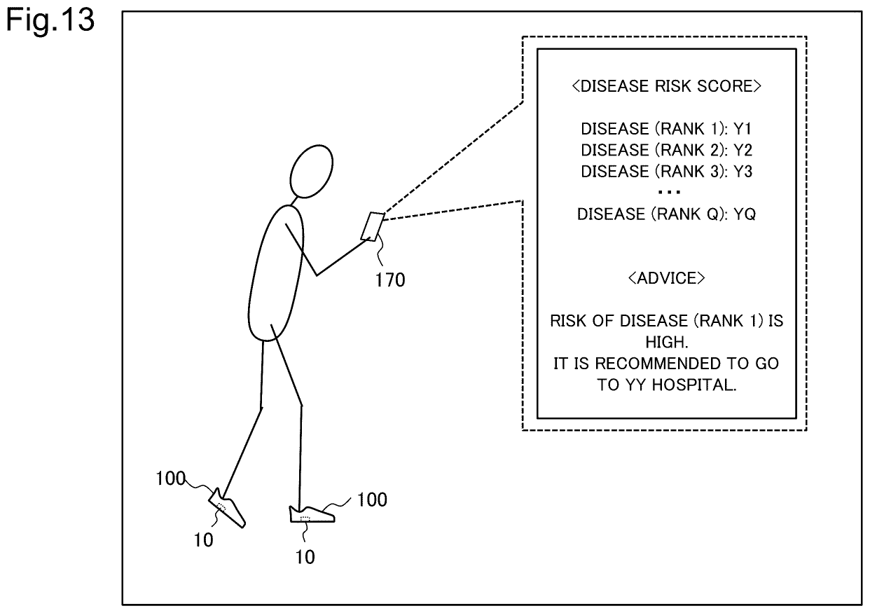

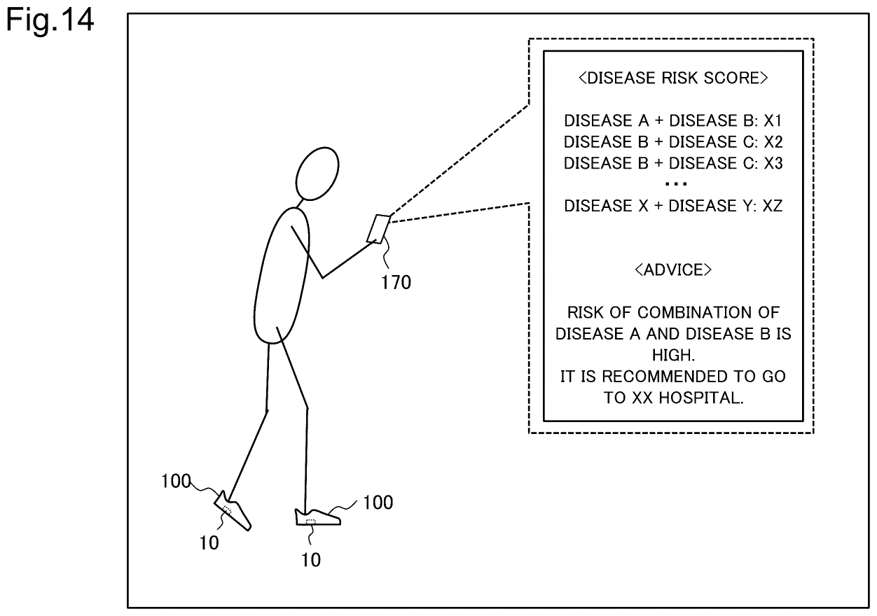

FIGS. 13 and 14 are conceptual diagrams illustrating an example in which the disease risk information estimated by the disease risk estimation device 13 is displayed on the screen of a mobile terminal 170 carried by the user who walks while wearing the shoe 100 on which the measurement device 10 is disposed. In the example of FIGS. 13 and 14, disease risk information estimated using sensor data measured while the user is walking is displayed on the screen of the mobile terminal 170. On the screen of the mobile terminal 170, disease risk information estimated for each user is optimized and displayed for each user. For example, the disease risk information includes advice according to a disease risk generated by being applied to a preset document format. For example, the advice according to the disease risk may be generated using a large-scale language model.

FIG. 13 illustrates an example in which the disease risk information including the disease risk score relevant to the rank indicating the risk of a disease is displayed on the screen of the mobile terminal 170. In the example of FIG. 13, the disease risk score reflecting a risk for each disease of “Disease (rank 1): Y1, Disease (rank 2): Y2, Disease (rank 3): Y3, . . . , Disease (rank Q): YQ” is displayed on the screen of the mobile terminal 170. In the example of FIG. 13, disease risk information including advice optimized for each user according to the disease risk of “There is a high risk of disease (rank 1). It is recommended to go to YY hospital for examination.” is displayed on the screen of the mobile terminal 170 according to the disease risk score.

FIG. 14 illustrates an example in which the disease risk information including the disease risk score relevant to the risk of the combination of diseases is displayed on the screen of the mobile terminal 170. In the example of FIG. 14, the disease risk score reflecting a risk for each disease combination of “Disease A+Disease B: X1, Disease B+Disease C: X2, Disease B+Disease C: X3, . . . , Disease X+Disease Y: XZ” is displayed on the screen of the mobile terminal 170. In the example of FIG. 14, disease risk information including advice according to a disease risk of “The risk of combination of disease A and disease B is high. It is recommended to go to XX hospital.” is displayed on the screen of the mobile terminal 170 according to the disease risk score.

In the above-described application example, the user who has confirmed the information related to the disease risk reflecting a risk for each disease displayed on the display unit of the mobile terminal 170 can recognize his/her disease risk. The disease risk information may be provided to a person other than the user. For example, the disease risk information may be output to a terminal device (not illustrated) used by a doctor or a trainer who performs physical condition management of the user, a family member of the user, or the like. For example, the disease risk information may be recorded in a database (not illustrated) constructed for the purpose of health management or the like. The output destination and use of the disease risk information are not particularly limited.

As described above, the disease risk estimation system according to the present example embodiment includes the measurement device and the disease risk estimation device. The measurement device is installed on the footwear of the subject as an estimation target of the disease risk information. The measurement device measures spatial acceleration and spatial angular velocity. The measurement device generates sensor data using the measured spatial acceleration and spatial angular velocity. The measurement device transmits the generated sensor data to the disease risk estimation device. The disease risk estimation device includes an acquisition unit, a risk estimation unit, and an output unit. The acquisition unit acquires sensor data measured in accordance with the movement of the foot of the subject who is an estimation target of the disease risk. The risk estimation unit includes a calculation unit and an estimation unit. The calculation unit calculates a gait index using the sensor data. The estimation unit inputs data including the gait index calculated using the sensor data to the disease risk estimation model. The disease risk estimation model outputs a disease risk score indicating a degree of the disease risk related to a disease according to an input of data including a gait index. The estimation unit estimates the disease risk reflecting a risk for each disease according to the disease risk score output from the disease risk estimation model. The output unit outputs disease risk information relevant to the estimated disease risk.

As described above, the disease risk estimation device according to the present example embodiment estimates the disease risk reflecting a risk for each disease using the sensor data measured in accordance with the sensor data related to the movement of the foot of the subject. That is, according to the present example embodiment, the disease risk reflecting a risk for each disease can be estimated using the sensor data measured according to the movement of the foot.

In one aspect of the present example embodiment, the estimation unit calculates the disease risk score reflecting a risk for each disease by multiplying the disease risk score by the weight for each disease according to the rank indicating the risk of the disease. According to the present aspect, it is possible to estimate the disease risk reflecting a risk for each disease according to the rank indicating the risk of the disease.

In one aspect of the present example embodiment, the estimation unit calculates the disease risk score reflecting a risk for each combination of diseases by multiplying the disease risk score by the weight for each combination of diseases. According to the present aspect, it is possible to estimate the disease risk score reflecting a risk for each combination of diseases.

In one aspect of the present example embodiment, the disease risk estimation device performs optimization for the subject and displaying on a screen of a terminal device browsable by the user. According to the present aspect, it is possible to perform optimization and provision for the subject.

In one aspect of the present example embodiment, the disease risk estimation model is a model learned using a machine learning method. For example, the disease risk estimation model includes an incomplete heterogeneous variational autoencoder. According to the present aspect, the disease risk of the subject can be estimated even if there is some loss in data such as the gait index.

### Second Example Embodiment

Next, a disease risk estimation device according to a second example embodiment will be described with reference to the drawings. The disease risk estimation device according to the present example embodiment outputs disease risk information including proposal information relevant to a change tendency of a disease risk.

### (Configuration)

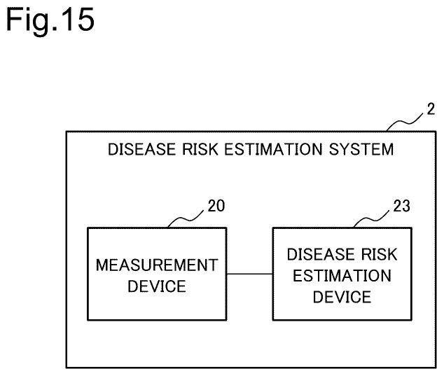

FIG. 15 is a block diagram illustrating an example of a configuration of a disease risk estimation system 2 in the present disclosure. The disease risk estimation system 2 includes a measurement device 20 and a disease risk estimation device 23. For example, the measurement device 20 is installed on footwear of a subject (user) who is an estimation target of the disease risk. For example, the function of the disease risk estimation device 23 is installed in a mobile terminal carried by the subject (user). The measurement device 20 has the same configuration as the measurement device 10 of the first example embodiment. Hereinafter, the description of the measurement device 20 will be omitted, and the disease risk estimation device 23 will be described. The main configuration of the disease risk estimation device 23 is similar to the configuration of the disease risk estimation device 13 of the first example embodiment, and thus the description thereof may be omitted.

### [Disease Risk Estimation Device]

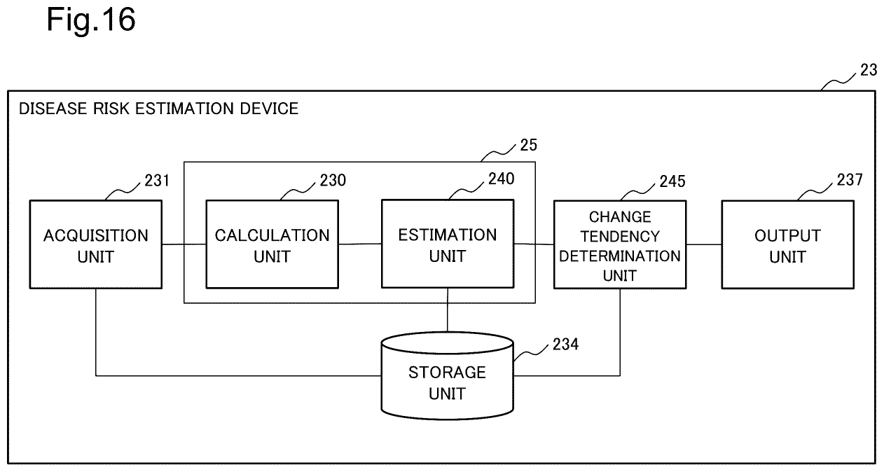

FIG. 16 is a block diagram illustrating an example of a configuration of the disease risk estimation device 23. The disease risk estimation device 23 includes an acquisition unit 231, a calculation unit 230, an estimation unit 240, a storage unit 234, a change tendency determination unit 245, and an output unit 237. The calculation unit 230 and the estimation unit 240 constitute a risk estimation unit 25.

The acquisition unit 231 (acquisition means) has the same configuration as the acquisition unit 131 of the first example embodiment. The acquisition unit 231 acquires sensor data from the measurement device 20. The acquisition unit 231 receives sensor data from the measurement device 20 via wireless communication. For example, the acquisition unit 231 receives sensor data from the measurement device 20 via a wireless communication function (not illustrated) conforming to a standard such as Bluetooth (registered trademark) or WiFi (registered trademark). The communication function of the acquisition unit 231 may conform to a standard other than Bluetooth (registered trademark) or WiFi (registered trademark) as long as the communication function can communicate with the measurement device 20. The acquisition unit 231 may receive the sensor data from the measurement device 20 via a wire such as a cable. For example, the acquisition unit 231 may acquire a gait index or a feature quantity calculated by the measurement device 20.

The acquisition unit 231 acquires body information (attribute) of the user. The body information includes gender, date of birth, height, and weight. The date of birth is converted to age. For example, the body information is input via an input device (not illustrated). For example, the body information is input via a mobile terminal used by the user. For example, the body information may be stored in the storage unit 234 in advance. The body information may be updated at an arbitrary timing according to an input by the user.

The calculation unit 230 (calculation means) has the same configuration as the calculation unit 130 of the first example embodiment.

The calculation unit 230 has the functions of the waveform processing unit 132 and the gait index calculation unit 133 of the first example embodiment. The calculation unit 230 acquires sensor data from the acquisition unit 231. The calculation unit 230 extracts time-series data (gait waveform data) for one gait cycle from the time-series data of the acceleration in the three-axis direction and the angular velocity around the three axes included in the sensor data. The calculation unit 230 extracts the gait waveform data based on the timing of the gait event detected from the time-series data of the sensor data. For example, the calculation unit 230 extracts the gait waveform data with the timing of the heel strike as a start point and the timing of the next heel strike as an end point.

The calculation unit 230 normalizes the time of the extracted gait waveform data for one gait cycle to a gait cycle of 0 to 100% (percent) (first normalization). The calculation unit 230 normalizes the gait waveform data subjected to the first normalization for one gait cycle so that the stance phase becomes 60% and the swing phase becomes 40% (second normalization).

The calculation unit 230 extracts a feature quantity (physical ability feature quantity) used to estimate the physical ability from the gait waveform data. The calculation unit 230 extracts a physical ability feature quantity used to estimate at least one physical ability. For example, the calculation unit 230 extracts a physical ability feature quantity used for estimation of at least one of physical abilities such as grip strength (total muscle strength of the whole body), dynamic balance, lower limb muscle strength, movement ability, and static balance. For example, the calculation unit 230 extracts the physical ability feature quantity for each gait phase cluster according to a preset condition. The calculation unit 230 outputs the extracted physical ability feature quantity to the estimation unit 240.

The calculation unit 230 calculates a gait index used for estimating the physical ability using the normalized gait waveform data. For example, the calculation unit 230 calculates a gait index related to a distance, a height, an angle, a speed, a time, a frailty level, a center of pressure exclusion index (CPEI), and the like.

The storage unit 234 (storage means) has the same configuration as the storage unit 134 of the first example embodiment. The storage unit 234 stores a physical ability estimation model for estimating the physical ability using the physical ability feature quantity extracted from the gait waveform data. For example, the physical ability estimation model outputs an index (physical ability score) related to the physical ability according to the input of the physical ability feature quantity extracted from the gait waveform data. The storage unit 234 stores a disease risk estimation model for estimating a disease risk using the body information, the gait index, and the physical ability score. For example, the disease risk estimation model outputs an index related to the disease risk (disease risk score) according to the input of the body information, the gait index, and the physical ability score.

The storage unit 234 stores the physical ability estimation model and the disease risk estimation model learned for a plurality of subjects. For example, the physical ability estimation model and the disease risk estimation model may be stored in the storage unit 234 at the time of factory shipment of a product. The physical ability estimation model and the disease risk estimation model may be stored in the storage unit 234 at a timing such as at the time of calibration before the user uses the disease risk estimation device 23. For example, a physical ability estimation model and a disease risk estimation model stored in a storage device (not illustrated) such as an external server may be used. In that case, it is sufficient that the physical ability estimation model and the disease risk estimation model can be accessed via an interface (not illustrated) connected to the storage device.

The storage unit 234 stores body information (attribute) of the user. The body information includes gender, date of birth, height, and weight. The date of birth is converted to age. The body information may be updated at any timing.

The estimation unit 240 (estimation means) has the same configuration as the estimation unit 140 of the first example embodiment. The estimation unit 240 includes the functions of the physical ability estimation unit 135 and the disease risk estimation unit 136 according to the first example embodiment. The estimation unit 240 acquires the physical ability feature quantity extracted from the gait waveform data from the calculation unit 230. The estimation unit 240 acquires the body information (attribute) stored in the storage unit 234. The estimation unit 240 estimates the physical ability score using the physical ability feature quantity and the body information (attribute). The estimation unit 240 inputs the physical ability feature quantity and the user's body information (attribute) to the physical ability estimation model stored in the storage unit 234. For example, the estimation unit 240 estimates a physical ability score related to the physical ability of at least one of grip strength (total muscle strength of the whole body), dynamic balance, lower limb muscle strength, movement ability, or static balance. The estimation unit 240 estimates the disease risk score reflecting a risk for each disease using the physical ability score, the gait index, and the body information (attribute). The estimation unit 240 outputs the estimated disease risk score.

The change tendency determination unit 245 acquires time-series data of the disease risk score reflecting a risk for each disease. The change tendency determination unit 245 determines a change tendency for each disease risk according to a change in the time-series data of the disease risk score. The change tendency determination unit 245 determines whether there is a change tendency such as an increasing tendency, a decreasing tendency, or a stagnation tendency in the time-series data of the disease risk score. The change tendency determination unit 245 determines the disease risk for each disease according to the change tendency for each disease risk.

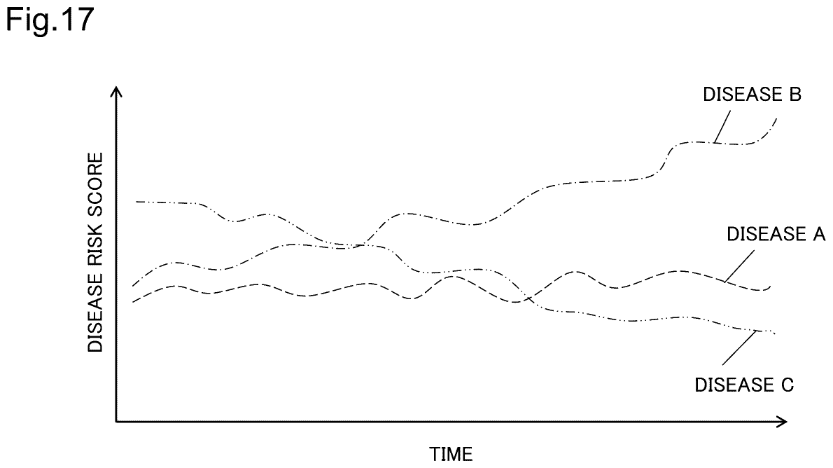

FIG. 17 is a graph illustrating an example of time-series data of the disease risk score related to one user. The graph of FIG. 17 relates to three diseases having different change tendencies of the disease risk scores. For example, the change tendency determination unit 245 determines a change tendency according to the slope of the disease risk score in the specific period. For example, the change tendency determination unit 245 may determine the change tendency according to the slope of the tangent fitted in accordance with the curve of the time-series data of the disease risk score.

In the example of FIG. 17, a stagnation tendency is observed with respect to the time-series data of the disease risk score of the disease A (broken line). Regarding the time-series data of the disease risk score of the disease B (one-dot chain line), an increasing tendency is observed. Regarding the time-series data of the disease risk score of the disease C (two-dot chain line), a decreasing tendency is observed. For example, the change tendency determination unit 245 determines that there is no change in the risk of the disease A according to the stagnation tendency of the disease risk score related to the disease A. For example, the change tendency determination unit 245 determines that there is a change in the risk of the disease B according to the increasing tendency of the disease risk score related to the disease B. For example, the change tendency determination unit 245 determines that the risk of the disease C has decreased according to the decreasing tendency of the disease risk score related to the disease C.

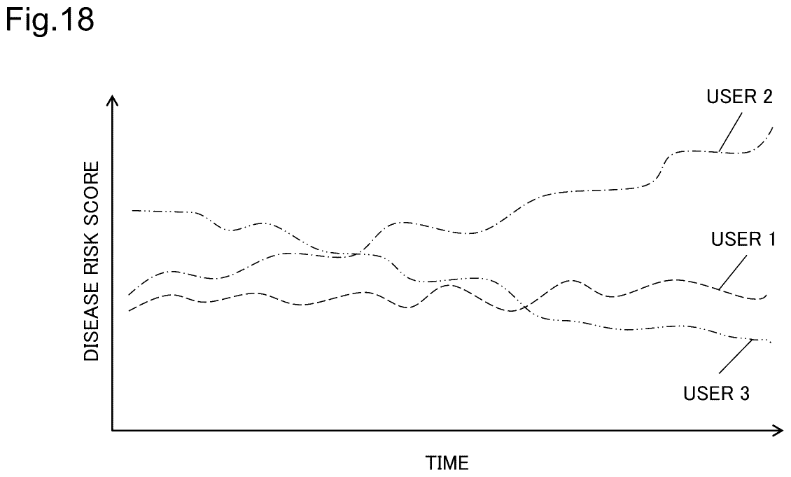

FIG. 18 is a graph illustrating an example of time-series data of disease risk scores related to a plurality of users. The graph of FIG. 18 relates to three users having different change tendencies of the same disease risk score. Regarding the time-series data of the disease risk score of the user 1 (broken line), a stagnation tendency is observed. Regarding the time-series data of the disease risk score of the user 2 (one-dot chain line), an increasing tendency is observed. Regarding the time-series data of the disease risk score of the user 3 (two-dot chain line), a decreasing tendency is observed. For example, the change tendency determination unit 245 determines that there is no change in the disease risk according to the stagnation tendency of the disease risk score related to the user 1. For example, the change tendency determination unit 245 determines that there is a change in the risk of a disease according to the increasing tendency of the disease risk score related to the user 2. For example, the change tendency determination unit 245 determines that the risk of a disease has decreased according to the decreasing tendency of the disease risk score related to the user 3.

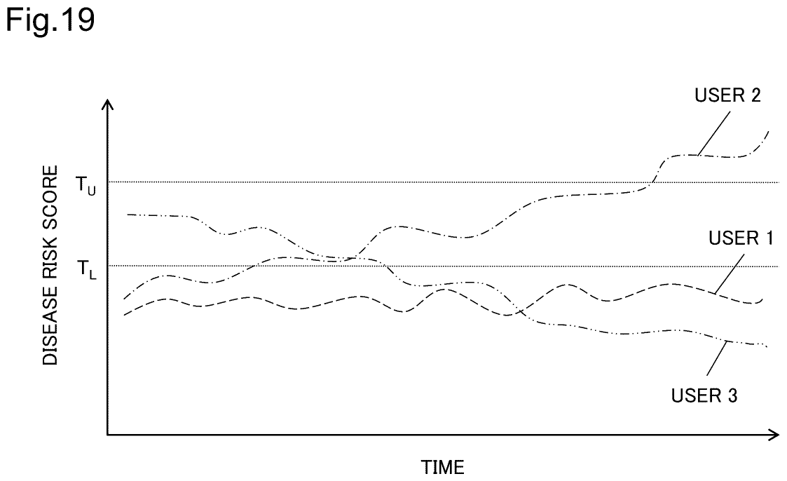

FIG. 19 is a graph illustrating an example of time-series data of disease risk scores related to a plurality of users. The graph of FIG. 19 relates to three users having different change tendencies of the same disease risk score. FIG. 19 illustrates an example in which a change tendency of the disease risk score is determined according to a threshold set to the disease risk score. In the example of FIG. 19, a lower limit threshold T_{L} and an upper limit threshold T_{U} are set. The time-series data of the disease risk score of the user 1 (broken line) is below the lower limit threshold T_{L}. The time-series data of the disease risk score of the user 2 (one-dot chain line) exceeds the lower limit threshold T_{L} and further exceeds the upper limit threshold T_{U}. The time-series data of the disease risk score of the user 3 (two-dot chain line) exceeds the lower limit threshold T_{L}, but falls below the lower limit threshold T_{L} as time passes. For example, the change tendency determination unit 245 determines that the risk of a disease is low since the disease risk score is below the lower limit threshold T_{L} for the user 1. For example, the change tendency determination unit 245 determines that the risk of a disease is high for the user 2 since the disease risk score exceeds the upper limit threshold T_{U}. For example, the change tendency determination unit 245 determines that the risk of disease has decreased for the user 3 since the disease risk score is below the lower limit threshold T_{L}. In the example of FIG. 19, two thresholds are set for the disease risk score. Only one threshold for the disease risk score may be set, or three or more thresholds may be set.

The change tendency determination unit 245 generates information relevant to the determination result related to the disease risk for each disease. For example, the change tendency determination unit 245 generates proposal information relevant to the determination result related to the disease risk for each disease. For example, the change tendency determination unit 245 generates proposal information including advice according to a disease risk generated by being applied to a preset document format. For example, the advice according to the disease risk may be generated using a large-scale language model.

The output unit 237 (output means) has the same configuration as the output unit 137 of the first example embodiment. The output unit 237 outputs disease risk information relevant to the disease risk score estimated by the estimation unit 240. The output unit 237 outputs the proposal information according to the determination result by the change tendency determination unit 245. For example, the output unit 237 displays the disease risk information and the proposal information on a screen of a mobile terminal of the subject (user). For example, the output unit 237 outputs the disease risk information and the proposal information to an external system or the like that uses the disease risk information and the proposal information. The use of the output disease risk information and the proposal information is not particularly limited. For example, the disease risk information and the proposal information are used for statistical analysis, disease prevention research, and the like.

### (Operation)

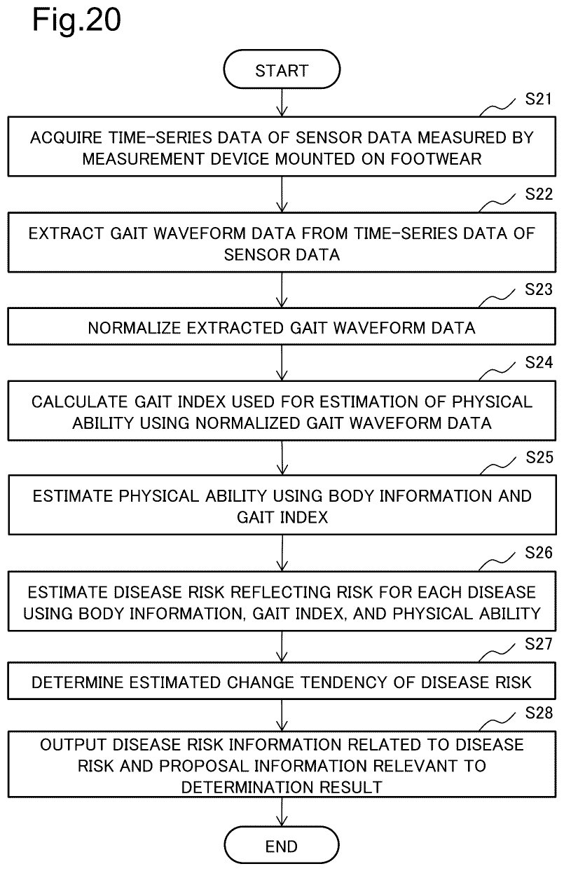

Next, the operation of the disease risk estimation system 2 will be described with reference to the drawings. Hereinafter, the operation of the disease risk estimation device 23 included in the disease risk estimation system 2 will be described. FIG. 20 is a flowchart for explaining an example of the operation of the disease risk estimation device 23. In the description of the processing along the flowchart of FIG. 20, the components of the disease risk estimation device 23 will be described as the operation subject. The operation subject of the processing along the flowchart of FIG. 20 may be the disease risk estimation device 23.

In FIG. 20, first, the acquisition unit 231 acquires time-series data of sensor data measured by the measurement device 20 mounted on the footwear (step S21). The sensor data includes acceleration in three axis directions and angular velocity around three axes.

Next, the calculation unit 230 extracts the gait waveform data from the time-series data of the sensor data (step S22). The gait waveform data is relevant to time-series data of sensor data for one gait cycle.

Next, the calculation unit 230 normalizes the extracted gait waveform data (step S23). The calculation unit 230 performs the first normalization on the gait waveform data in one gait cycle of 100%. The calculation unit 230 performs the second normalization on the gait waveform data so that the stance phase becomes 60% and the swing phase becomes 40%.

Next, the calculation unit 230 calculates a gait index used for estimating the physical ability using the normalized gait waveform data (step S24). For example, the calculation unit 230 calculates a gait index related to a distance, a height, an angle, a speed, a time, a frailty level, CPEI, and the like.

Next, the estimation unit 240 estimates the physical ability using the body information and the gait index (step S25). For example, the estimation unit 240 estimates physical ability scores such as grip strength (total muscle strength of the whole body), dynamic balance, lower limb muscle strength, movement ability, and static balance.

Next, the estimation unit 240 estimates the disease risk reflecting a risk for each disease using the body information, the gait index, and the physical ability (step S26). The estimation unit 240 estimates a disease risk score reflecting a risk for each disease. For example, the estimation unit 240 estimates a disease risk score reflecting a risk for each disease such as gout, diabetes, hypertension, nephrolithiasis, liver cirrhosis, arteriosclerosis, thromboembolism, dyslipidemia, hypercholesterolemia, or hyperlipidemia. For example, the estimation unit 240 estimates a disease risk score reflecting a risk for each disease such as low back pain, sleep apnea syndrome, insomnia, depression, knee osteoarthritis, and Parkinson's syndrome.

Next, the change tendency determination unit 245 determines a change tendency of the estimated disease risk (step S27). The change tendency determination unit 245 generates proposal information according to a change tendency related to the time-series data of the disease risk score.

Next, the output unit 237 outputs disease risk information related to the estimated disease risk and proposal information relevant to the determination result (step S28). For example, the output unit 237 displays the disease risk information and the proposal information on a screen of a mobile terminal of the subject (user). For example, the output unit 237 outputs the disease risk information and the proposal information to an external system or the like that uses the disease risk information and the proposal information.

### Application Example

Next, an application example according to the present example embodiment will be described with reference to the drawings. In the following application example, an example of estimating the disease risk using the feature quantity data measured by the measurement device 20 arranged in the shoe will be described. For example, the function of the disease risk estimation device 23 is installed in a mobile terminal carried by a user. The function of the disease risk estimation device 23 may be implemented in a server or a cloud connected to a mobile terminal carried by the user in a data-communicable manner.

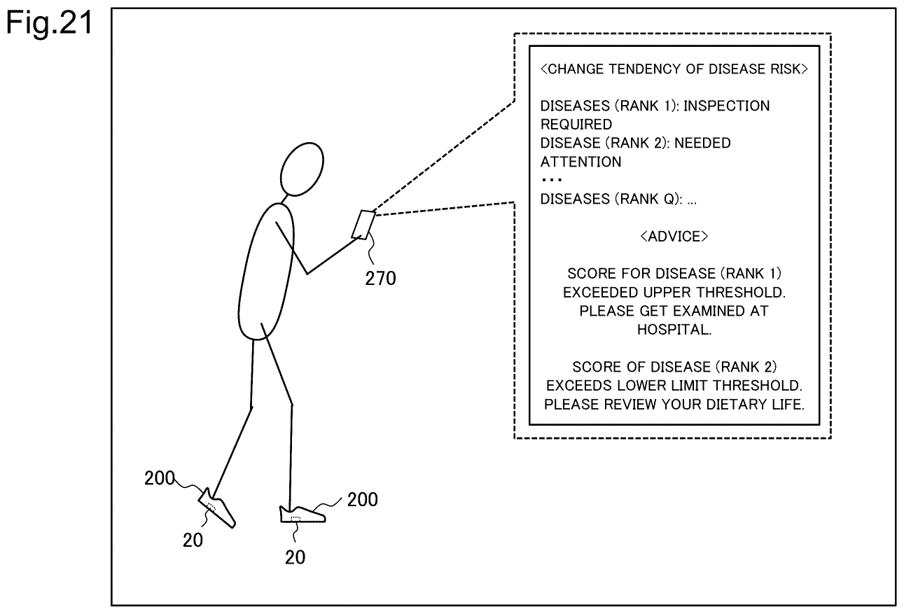

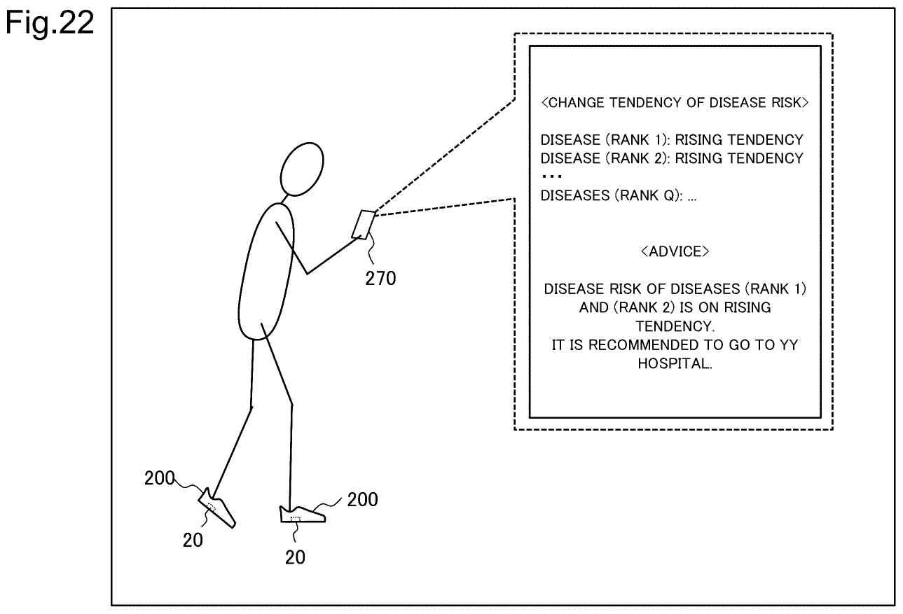

FIGS. 21 and 22 are conceptual diagrams illustrating an example in which the disease risk information estimated by the disease risk estimation device 23 is displayed on the screen of a mobile terminal 270 carried by the user who walks while wearing the shoe 200 on which the measurement device 20 is disposed. In the example of FIGS. 21 and 22, disease risk information estimated using sensor data measured while the user is walking is displayed on the screen of the mobile terminal 270. On the screen of the mobile terminal 270, disease risk information estimated for each user is optimized and displayed for each user. For example, the disease risk information includes advice according to a disease risk generated by being applied to a preset document format. For example, the advice according to the disease risk may be generated using a large-scale language model.

FIG. 21 illustrates an example in which the disease risk information including the change tendency of the disease risk and the advice is displayed on the screen of the mobile terminal 270. In the case of the example of FIG. 21, information relevant to the change tendency of the disease risk of “Disease (rank 1): inspection required, Disease (rank 2): caution required, . . . , Disease (rank Q): . . . ” is displayed on the screen of the mobile terminal 270. In the example of FIG. 21, disease risk information including advice according to a change tendency of a disease risk of “The score of Disease (rank 1) exceed upper threshold. Please get examined at the hospital.” is displayed on the screen of the mobile terminal 270 according to a change tendency of a disease risk requiring inspection. Furthermore, in the example of FIG. 21, disease risk information including advice according to the disease risk of “The score of Disease (rank 2) exceeds lower limit threshold. Please review your dietary life.” is displayed on the screen of the mobile terminal 270 according to the change tendency of the disease risk that requires attention.

FIG. 22 illustrates an example in which the disease risk information including the change tendency of the disease risk and the advice is displayed on the screen of the mobile terminal 270. In the case of the example of FIG. 22, information relevant to the change tendency of the disease risk of “Disease (rank 1): rising tendency, Disease (rank 2): rising tendency, . . . , Disease (rank Q): . . . ” is displayed on the screen of the mobile terminal 270. In the example of FIG. 22, disease risk information including advice according to the disease risk of “The disease risk of Disease (rank 1) and Disease (rank 2) is on a rising tendency. It is recommended to go to YY hospital.” is displayed on the screen of the mobile terminal 270 according to the change tendency of the disease risk.

In the above-described application example, the user who has confirmed the disease risk information relevant to the change tendency of the disease risk displayed on the display unit of the mobile terminal 270 can recognize his/her disease risk. The disease risk information may be provided to a person other than the user. For example, the disease risk information may be output to a terminal device (not illustrated) used by a doctor or a trainer who performs physical condition management of the user, a family member of the user, or the like. For example, the disease risk information may be recorded in a database (not illustrated) constructed for the purpose of health management or the like. The output destination and use of the disease risk information are not particularly limited.

As described above, the disease risk estimation system according to the present example embodiment includes the measurement device and the disease risk estimation device. The measurement device is installed on the footwear of the subject as an estimation target of the disease risk information. The measurement device measures spatial acceleration and spatial angular velocity. The measurement device generates sensor data using the measured spatial acceleration and spatial angular velocity. The measurement device transmits the generated sensor data to the disease risk estimation device. The disease risk estimation device includes an acquisition unit, a risk estimation unit, a change tendency determination unit, and an output unit. The acquisition unit acquires sensor data measured in accordance with the movement of the foot of the subject who is an estimation target of the disease risk. The risk estimation unit includes a calculation unit and an estimation unit. The calculation unit calculates a gait index using the sensor data. The estimation unit inputs data including the gait index calculated using the sensor data to the disease risk estimation model. The disease risk estimation model outputs a disease risk score indicating a degree of the disease risk related to a disease according to an input of data including a gait index. The estimation unit estimates the disease risk reflecting a risk for each disease according to the disease risk score output from the disease risk estimation model. The change tendency determination unit determines the disease risk for each disease according to the change tendency of the disease risk score. The output unit outputs disease risk information relevant to the estimated disease risk.

As described above, the disease risk estimation device according to the present example embodiment estimates the disease risk reflecting a risk for each disease using the sensor data measured in accordance with the sensor data related to the movement of the foot of the subject. The disease risk estimation device according to the present example embodiment determines the disease risk for each disease according to the change tendency of the disease risk score. That is, according to the present example embodiment, the disease risk reflecting a risk for each disease can be estimated according to the change tendency of the disease risk score.

In one aspect of the present example embodiment, the change tendency determination unit determines that the disease risk is high with respect to a disease for which the disease risk score is on an increasing tendency. The change tendency determination unit determines that the disease risk is low with respect to any disease for which the disease risk score is on any one of the decreasing tendency and the stagnation tendency. According to the present aspect, the disease risk can be determined according to the increasing tendency, the decreasing tendency, and the stagnation tendency of the disease risk score.

In one aspect of the present example embodiment, the change tendency determination unit determines that the disease risk is high with respect to a disease for which a disease risk score exceeds a threshold.

The change tendency determination unit determines that the disease risk is low with respect to a disease for which the disease risk score is below the threshold. According to the present aspect, the disease risk can be determined based on the relationship between the disease risk score and the threshold.

### Third Example Embodiment

Next, a disease risk estimation device according to a third example embodiment will be described with reference to the drawings. The disease risk estimation device according to the present example embodiment outputs disease risk information including proposal information relevant to a disease risk. The disease risk estimation device according to the present example embodiment outputs proposal information for an insurance-related institution such as a health insurance union or a life insurance company.

### (Configuration)

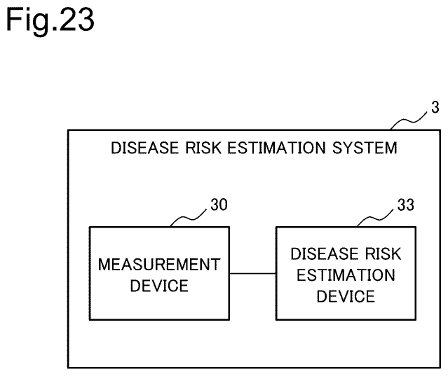

FIG. 23 is a block diagram illustrating an example of a configuration of a disease risk estimation system 3 in the present disclosure. The disease risk estimation system 3 includes a measurement device 30 and a disease risk estimation device 33. For example, the measurement device 30 is installed on the footwear of the subject who is an estimation target of a disease risk. For example, the function of the disease risk estimation device 33 is installed in a mobile terminal carried by the subject. The measurement device 30 has the same configuration as the measurement device 10 of the first example embodiment. Hereinafter, the description of the measurement device 30 will be omitted, and the disease risk estimation device 33 will be described. The main configuration of the disease risk estimation device 33 is similar to the configuration of the disease risk estimation device 13 of the first example embodiment, and thus the description thereof may be omitted.

### [Disease Risk Estimation Device]

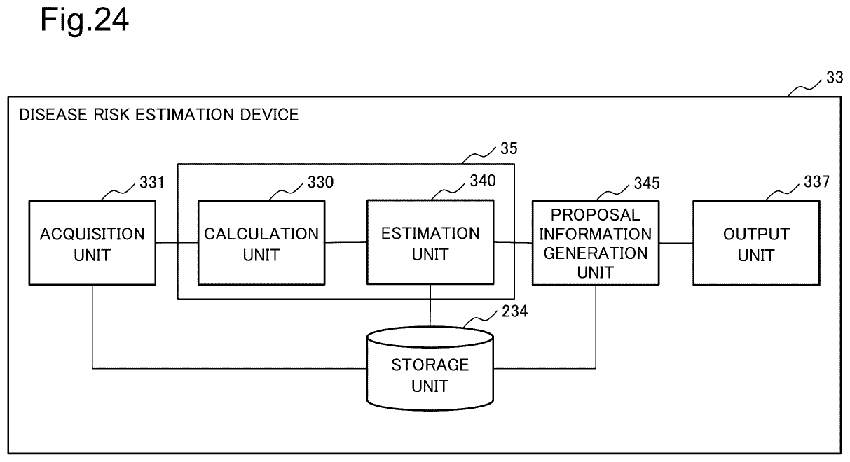

FIG. 24 is a block diagram illustrating an example of a configuration of the disease risk estimation device 33. The disease risk estimation device 33 includes an acquisition unit 331, a calculation unit 330, an estimation unit 340, a storage unit 334, a proposal information generation unit 345, and an output unit 337. The calculation unit 330 and the estimation unit 340 constitute a risk estimation unit 35. FIG. 24 illustrates a configuration in which the proposal information generation unit 345 is added to the configuration of the first example embodiment. The proposal information generation unit 345 may be added to the configuration of the second example embodiment.

The acquisition unit 331 (acquisition means) has the same configuration as the acquisition unit 131 of the first example embodiment. The acquisition unit 331 acquires sensor data from the measurement device 30. The acquisition unit 331 receives sensor data from the measurement device 30 via wireless communication. For example, the acquisition unit 331 receives sensor data from the measurement device 30 via a wireless communication function (not illustrated) conforming to a standard such as Bluetooth (registered trademark) or WiFi (registered trademark). The communication function of the acquisition unit 331 may conform to a standard other than Bluetooth (registered trademark) or WiFi (registered trademark) as long as the communication function can communicate with the measurement device 30. The acquisition unit 331 may receive the sensor data from the measurement device 30 via a wire such as a cable. For example, the acquisition unit 331 may acquire a gait index or a feature quantity calculated by the measurement device 30.

The acquisition unit 331 acquires body information (attribute) of the user. The body information includes gender, date of birth, height, and weight. The date of birth is converted to age. For example, the body information is input via an input device (not illustrated). For example, the body information is input via a mobile terminal used by the user. For example, the body information may be stored in the storage unit 334 in advance. The body information may be updated at an arbitrary timing according to an input by the user.

The calculation unit 330 (calculation means) has the same configuration as the calculation unit 130 of the first example embodiment. The calculation unit 330 has the functions of the waveform processing unit 132 and the gait index calculation unit 133 of the first example embodiment. The calculation unit 330 acquires sensor data from the acquisition unit 331. The calculation unit 330 extracts time-series data (gait waveform data) for one gait cycle from the time-series data of the acceleration in the three-axis direction and the angular velocity around the three axes included in the sensor data. The calculation unit 330 extracts the gait waveform data based on the timing of the gait event detected from the time-series data of the sensor data. For example, the calculation unit 330 extracts the gait waveform data with the timing of the heel strike as a start point and the timing of the next heel strike as an end point.

The calculation unit 330 normalizes the time of the extracted gait waveform data for one gait cycle to a gait cycle of 0 to 100% (percent) (first normalization). The calculation unit 330 normalizes the gait waveform data subjected to the first normalization for one gait cycle so that the stance phase becomes 60% and the swing phase becomes 40% (second normalization).

The calculation unit 330 extracts a feature quantity (physical ability feature quantity) used to estimate the physical ability from the gait waveform data. The calculation unit 330 extracts a physical ability feature quantity used to estimate at least one physical ability. For example, the calculation unit 330 extracts a physical ability feature quantity used for estimation of at least one of physical abilities such as grip strength (total muscle strength of the whole body), dynamic balance, lower limb muscle strength, movement ability, and static balance. For example, the calculation unit 330 extracts the physical ability feature quantity for each gait phase cluster according to a preset condition. The calculation unit 330 outputs the extracted physical ability feature quantity to the estimation unit 340.

The calculation unit 330 calculates a gait index used for estimating the physical ability using the normalized gait waveform data. For example, the calculation unit 330 calculates a gait index related to a distance, a height, an angle, a speed, a time, a frailty level, a center of pressure exclusion index (CPEI), and the like.

The storage unit 334 (storage means) has the same configuration as the storage unit 134 of the first example embodiment. The storage unit 334 stores a physical ability estimation model for estimating the physical ability using the physical ability feature quantity extracted from the gait waveform data. For example, the physical ability estimation model outputs an index (physical ability score) related to the physical ability according to the input of the physical ability feature quantity extracted from the gait waveform data. The storage unit 334 stores a disease risk estimation model for estimating a disease risk using the body information, the gait index, and the physical ability score. For example, the disease risk estimation model outputs an index related to the disease risk (disease risk score) according to the input of the body information, the gait index, and the physical ability score.

The storage unit 334 stores the physical ability estimation model and the disease risk estimation model learned for a plurality of subjects. For example, the physical ability estimation model and the disease risk estimation model may be stored in the storage unit 334 at the time of factory shipment of a product. The physical ability estimation model and the disease risk estimation model may be stored in the storage unit 334 at a timing such as at the time of calibration before the subject uses the disease risk estimation device 33. For example, a physical ability estimation model and a disease risk estimation model stored in a storage device (not illustrated) such as an external server may be used. In that case, it is sufficient that the physical ability estimation model and the disease risk estimation model can be accessed via an interface (not illustrated) connected to the storage device.

The storage unit 334 stores body information (attribute) of the subject. The body information includes gender, date of birth, height, and weight. The date of birth is converted to age. The body information may be updated at any timing.

The estimation unit 340 (estimation means) has the same configuration as the estimation unit 140 of the first example embodiment. The estimation unit 340 includes the functions of the physical ability estimation unit 135 and the disease risk estimation unit 136 according to the first example embodiment. The estimation unit 340 acquires the physical ability feature quantity extracted from the gait waveform data from the calculation unit 330. The estimation unit 340 acquires the body information (attribute) stored in the storage unit 334. The estimation unit 340 estimates the physical ability score using the physical ability feature quantity and the body information (attribute). The estimation unit 340 inputs the physical ability feature quantity and the subject's body information (attribute) to the physical ability estimation model stored in the storage unit 334. For example, the estimation unit 340 estimates a physical ability score related to the physical ability of at least one of grip strength (total muscle strength of the whole body), dynamic balance, lower limb muscle strength, movement ability, or static balance. The estimation unit 340 estimates the disease risk score reflecting a risk for each disease using the physical ability score, the gait index, and the body information (attribute). The estimation unit 340 outputs the estimated disease risk score.

The proposal information generation unit 345 acquires a disease risk score reflecting a risk for each disease. The proposal information generation unit 345 generates proposal information for an insurance-related institution such as a health insurance union or a life insurance company according to the disease risk score. For example, the proposal information generation unit 345 generates disease risk information including proposal information related to a subject who is an insured person for an insurance-related institution such as a health insurance union or a life insurance company. In response to acquisition of the disease risk information, the health insurance union or the life insurance company can take an action relevant to the proposal information for the subject who is an insured person.

The proposal information generation unit 345 generates proposal information relevant to the disease risk score for each disease. For example, the proposal information generation unit 345 may generate proposal information according to a change tendency of the disease risk score for each disease. For example, the proposal information generation unit 345 generates proposal information including advice according to a disease risk score generated by being applied to a preset document format. For example, the advice according to the disease risk score may be generated using a large-scale language model.

The output unit 337 (output means) has the same configuration as the output unit 137 of the first example embodiment. The output unit 337 outputs disease risk information relevant to the disease risk score estimated by the estimation unit 340. The output unit 337 outputs proposal information according to the disease risk score by the proposal information generation unit 345. For example, the output unit 337 outputs the disease risk information and the proposal information to an external system or the like that uses the disease risk information and the proposal information. For example, the output unit 337 outputs disease risk information and proposal information to a terminal device (not illustrated) used in an insurance-related institution such as a health insurance union or a life insurance company. In this case, an insurance-related institution such as a health insurance union or a life insurance company is relevant to the user who browses the disease risk information or the proposal information. The use of the output disease risk information and the proposal information is not particularly limited. For example, the disease risk information and the proposal information are used for determining the timing at which the health insurance union provides specific health guidance to the subject. For example, the disease risk information and the proposal information are used to determine the timing at which the life insurance company gives an incentive such as a discount of insurance premiums or a benefit to the subject. For example, the disease risk information and the proposal information may be used for statistical analysis, disease prevention research, and the like.

### (Operation)

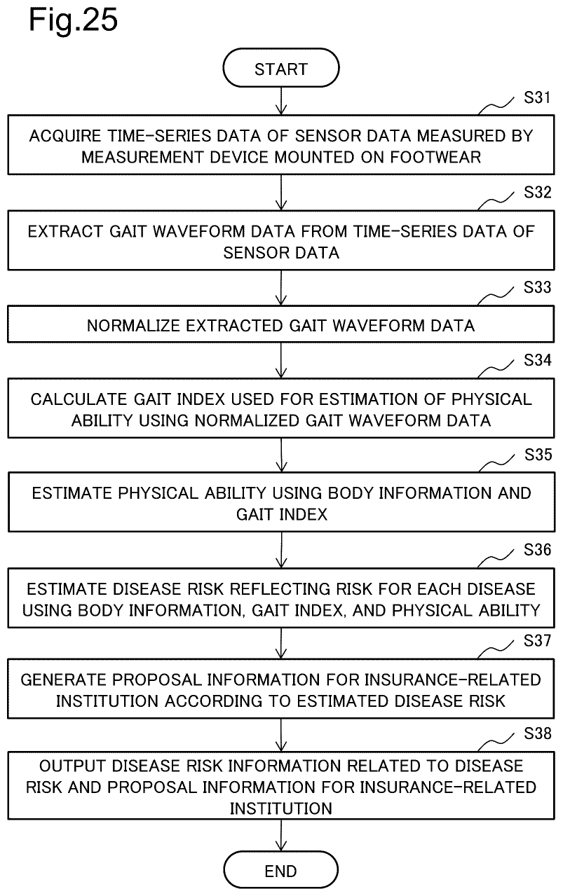

Next, the operation of the disease risk estimation system 3 will be described with reference to the drawings. Hereinafter, the operation of the disease risk estimation device 33 included in the disease risk estimation system 3 will be described. FIG. 25 is a flowchart for explaining an example of the operation of the disease risk estimation device 33. In the description of the processing along the flowchart of FIG. 25, the components of the disease risk estimation device 33 will be described as the operation subject. The operation subject of the processing along the flowchart of FIG. 25 may be the disease risk estimation device 33.

In FIG. 25, first, the acquisition unit 331 acquires time-series data of sensor data measured by the measurement device 30 mounted on the footwear (step S31). The sensor data includes acceleration in three axis directions and angular velocity around three axes.

Next, the calculation unit 330 extracts the gait waveform data from the time-series data of the sensor data (step S32). The gait waveform data is relevant to time-series data of sensor data for one gait cycle.

Next, the calculation unit 330 normalizes the extracted gait waveform data (step S33). The calculation unit 330 performs the first normalization on the gait waveform data in one gait cycle of 100%. The calculation unit 330 performs the second normalization on the gait waveform data so that the stance phase becomes 60% and the swing phase becomes 40%.

Next, the calculation unit 330 calculates a gait index used for estimating the physical ability using the normalized gait waveform data (step S34). For example, the calculation unit 330 calculates a gait index related to a distance, a height, an angle, a speed, a time, a frailty level, CPEI, and the like.

Next, the estimation unit 340 estimates the physical ability using the body information and the gait index (step S35). For example, the estimation unit 340 estimates physical ability scores such as grip strength (total muscle strength of the whole body), dynamic balance, lower limb muscle strength, movement ability, and static balance.

Next, the estimation unit 340 estimates the disease risk reflecting a risk for each disease using the body information, the gait index, and the physical ability (step S36). The estimation unit 340 estimates a disease risk score reflecting a risk for each disease. For example, the estimation unit 340 estimates a disease risk score reflecting a risk for each disease such as gout, diabetes, hypertension, nephrolithiasis, liver cirrhosis, arteriosclerosis, thromboembolism, dyslipidemia, hypercholesterolemia, or hyperlipidemia. For example, the estimation unit 340 estimates a disease risk score reflecting a risk for each disease such as low back pain, sleep apnea syndrome, insomnia, depression, knee osteoarthritis, and Parkinson's syndrome.

Next, the proposal information generation unit 345 generates proposal information for an insurance-related institution such as a health insurance union or a life insurance company according to the estimated disease risk (step S37).

Next, the output unit 337 outputs disease risk information related to a disease risk and proposal information for an insurance-related institution (step S38). For example, the disease risk information and the proposal information are used for determining the timing at which the health insurance union provides specific health guidance to the subject. For example, the disease risk information and the proposal information are used to determine the timing at which the life insurance company gives an incentive such as a discount of insurance premiums or a benefit to the subject. For example, the disease risk information and the proposal information may be used for statistical analysis, disease prevention research, and the like.

### Application Example

Next, an application example according to the present example embodiment will be described with reference to the drawings. In the following application example, a relationship among a business operator providing a service using the disease risk estimation system 3, an insurance-related institution using the service, and a subject who receives a benefit or the like from the insurance-related institution is illustrated. Hereinafter, examples of the health insurance union and the life insurance company will be individually described.

### [Health Insurance Union]

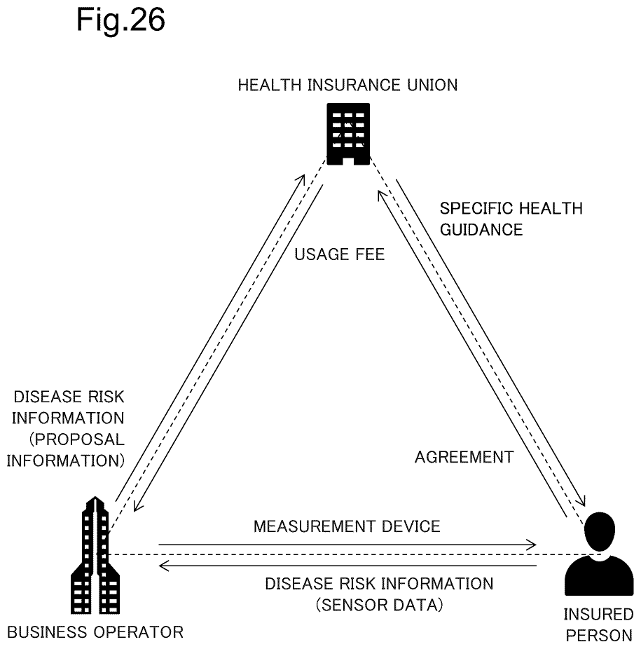

FIG. 26 illustrates an example in which an insurance-related institution is a health insurance union. The business operator provides a service using the disease risk estimation system 3 to the health insurance union. Based on the contract concluded with the health insurance union, the business operator provides the health insurance union with disease risk information and proposal information related to the insured person. The health insurance union pays a service usage fee for the disease risk estimation system 3 to the business operator. In the contract between the business operator and the health insurance union, rules related to handling of personal information and appropriate data management are clarified. The business operator clearly explains that the disease risk score is reference information and does not guarantee medical accuracy or completeness.

The health insurance union sufficiently explains the contents of the personal information protection policy and the data management to the insured person, and then obtains consent from the insured person. In a case where there is a change in the personal information protection policy or the content of data management, the health insurance union explains the change to the insured person and obtains consent from the insured person. For example, consent from the insured person is performed electronically. The health insurance union provides specific health guidance to the insured person according to the content of the disease risk information provided by the business operator.

The insured person is a subject who pays the health insurance premium to the health insurance union. The insured person receives a loan or donation of a dedicated insole equipped with the measurement device 30 from a business operator who has a contract with the health insurance union. The business operator provides support related to a method of installing and operating a dedicated application having the function of the disease risk estimation device 33, a method of wearing a dedicated insole, and the like. The business operator cooperates with the health insurance union to add or update new functions and provide appropriate support to the insured person. The insured person installs a dedicated application having the function of the disease risk estimation device 33 on his/her mobile terminal. The function of the disease risk estimation device 33 may be installed in a cloud server managed by the business operator. The insured person walks carrying a mobile terminal with a dedicated application installed, wearing shoes with a dedicated insole. The dedicated application estimates the disease risk score using the sensor data measured by the measurement device 30 according to the gait of the insured person. The dedicated application uploads data including disease risk information relevant to the disease risk score to the cloud server of the business operator. The dedicated application may upload the sensor data measured by the measurement device 30 to a cloud server managed by the business operator. For example, the dedicated application displays disease risk information relevant to the disease risk score estimated by the disease risk estimation device 33 on a screen of a mobile terminal carried by the insured person. For example, the insured person improves the lifestyle according to the improvement advice included in the disease risk information.

A terminal device used in a health insurance union downloads disease risk information of an insured person from a cloud server of a business operator. The health insurance union refers to disease risk information. The health insurance union provides specific health guidance to the insured person according to the disease risk score included in the disease risk information. The specific health guidance is performed by an expert with specialized knowledge. The health insurance union periodically refers to the disease risk score of the insured person, and provides health support and counseling by an expert according to a change in the score. For example, the health insurance union holds regular consultation meetings and events, and provides information related to health maintenance using the measurement device 30. The health insurance union takes in the opinions and demands of the insured person. The health insurance union periodically verifies and evaluates whether the health improvement and the medical cost reduction of the insured person have been implemented by applying the disease risk estimation system 3.

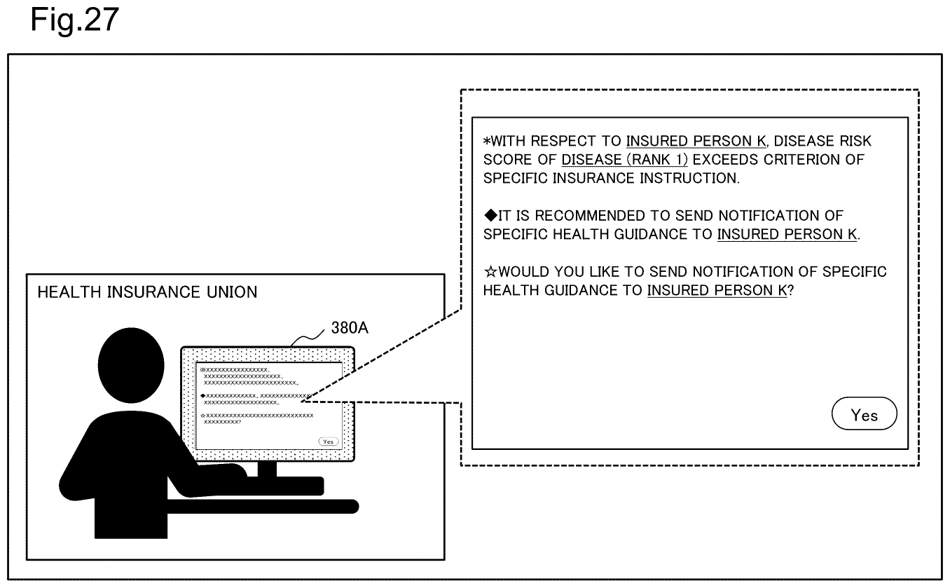

FIG. 27 illustrates an example in which the disease risk information related to the insured person is displayed on the screen of a terminal device 380A used in the health insurance union. On the screen of the terminal device 380A, information relevant to the disease risk score of “With respect to the insured person K, the disease risk of the disease (rank 1) exceeds a criterion of the specific health guidance.” is displayed. Further, proposal information “It is recommended to send a notification of specific health guidance to the insured person K.” is displayed on the screen of the terminal device 380A. This proposal information is displayed at an appropriate timing when the health insurance union notifies the insured person of the specific health guidance. Further, proposal information “Would you like to send a notification of specific health guidance to the insured person K?” is displayed on the screen of the terminal device 380A. A clerical staff who has confirmed the disease risk information displayed on the screen of the terminal device 380A can notify the insured person of the specific health guidance. For example, when a “YES” button displayed on the screen of the terminal device 380A is pressed, a notification of specific health guidance is transmitted to a mobile terminal carried by the insured person.

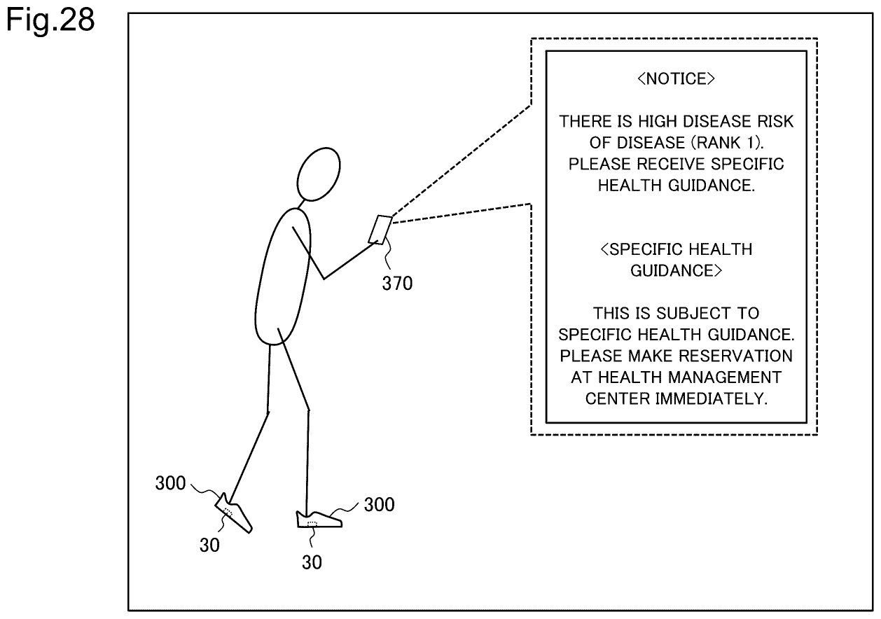

FIG. 28 illustrates an example in which the information relevant to the notification of the specific health guidance transmitted from the terminal device 380A of the health insurance union is displayed on the screen of the mobile terminal 370 carried by the insured person walking while wearing the shoes 300 on which the measurement device 30 is disposed. In the example of FIG. 28, a notification “There is high disease risk of the disease (rank 1). Please receive specific health guidance.” is displayed on the screen of the mobile terminal 370. In the example of FIG. 28, disease risk information including advice related to specific health guidance of “This is subject to specific health guidance. Please make a reservation at the health management center immediately.” is displayed on the screen of the mobile terminal 370 according to the disease risk score. The disease risk information may include statistical information of all insured persons. For example, the ratio of the insured person whose disease risk score has been improved by receiving the specific health guidance among other insured persons whose disease risk scores are similar to those of the insured person who has been notified of the specific health guidance may be presented. Specifically, guidance such as “70% of people with a disease risk similar to you have improved the disease risk by receiving the specific insurance instruction.” is included in the advice related to specific health guidance. As a result, the insured person can perform his/her health management while referring to the tendency of a person in a state similar to his/her own.

The insured person who has confirmed the information relevant to the notification of the specific health guidance displayed on the display unit of the mobile terminal 370 can recognize that it is necessary to receive the specific health guidance. The notification of the specific health guidance may be provided to a person other than the insured person. For example, the notification of the specific health guidance may be transmitted to a terminal device (not illustrated) used by a doctor or a trainer who manages the physical condition of the insured person, a family member of the insured person, a supervisor of the company of the insured person, or the like. For example, the notification of the specific health guidance may be recorded in a database (not illustrated) constructed for the purpose of health management or the like. The transmission destination and use of the notification of specific health guidance are not particularly limited.

### [Life Insurance Company]

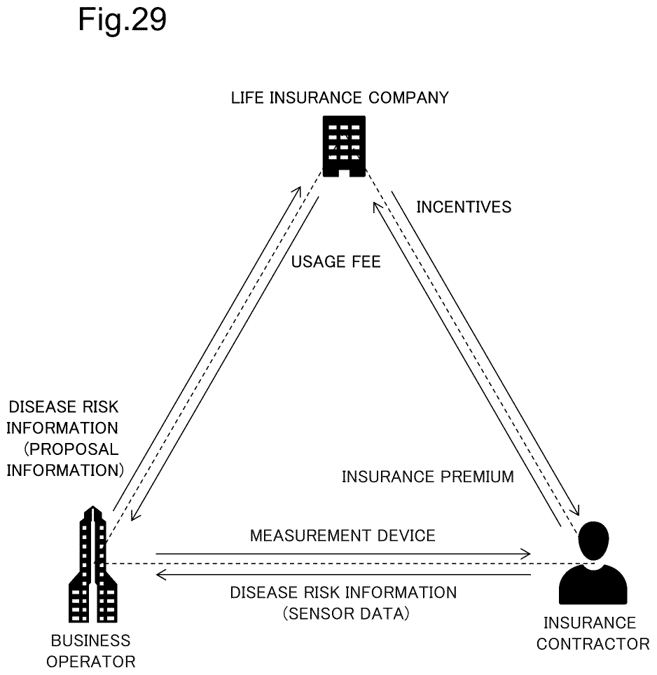

FIG. 29 illustrates an example in which the insurance-related institution is a life insurance company. The business operator provides a service using the disease risk estimation system 3 to a life insurance company. Based on the contract concluded with the life insurance company, the business operator provides the life insurance company with disease risk information and proposal information related to the insurance contractor. The life insurance company pays a service usage fee for the disease risk estimation system 3 to the business operator. In the contract between the business operator and the life insurance company, rules related to handling of personal information and appropriate data management are clarified. The business operator clearly explains that the disease risk score is reference information and does not guarantee medical accuracy or completeness.

The life insurance company sufficiently explains the contents of the personal information protection policy and the data management to the insurance contractor, and then obtains consent from the insurance contractor. In a case where there is a change in the personal information protection policy or the content of data management, the life insurance company explains the change to the insurance contractor and obtains consent from the insurance contractor. For example, agreement from an insurance contractor is implemented electronically. The life insurance company gives an incentive to the insurance contractor according to the content of the disease risk information provided from the business operator.

The insurance contractor is a subject that pays insurance premiums to the life insurance company. The insurance contractor is lent or offered a dedicated insole equipped with the measurement device 30 from a business operator who has a contract with a life insurance company. The business operator provides support related to a method of installing and operating a dedicated application having the function of the disease risk estimation device 33, a method of wearing a dedicated insole, and the like. The business operator cooperates with the health insurance union to add or update new functions and provide appropriate support to the insurance contractor. The insurance contractor installs a dedicated application having the function of the disease risk estimation device 33 on his/her mobile terminal. The function of the disease risk estimation device 33 may be installed in a cloud server managed by the business operator. The insurance contractor walks carrying a mobile terminal with a dedicated application installed, wearing shoes with a dedicated insole. The dedicated application estimates the disease risk score using the sensor data measured by the measurement device 30 according to the gait of the insurance contractor. The dedicated application uploads data including disease risk information relevant to the disease risk score to the cloud server of the business operator. The dedicated application may upload the sensor data measured by the measurement device 30 to a cloud server managed by the business operator. For example, the dedicated application displays disease risk information relevant to the disease risk score estimated by the disease risk estimation device 33 on a screen of a mobile terminal carried by the insurance contractor. For example, the insurance contractor improves the lifestyle according to the improvement advice included in the disease risk information.

A terminal device used in a life insurance company downloads disease risk information of an insurance contractor from a cloud server of a business operator. The health insurance union refers to disease risk information. The life insurance company gives an incentive to the insurance contractor according to the disease risk score included in the disease risk information. For example, a life insurance company gives an incentive such as a discount of insurance premiums or a benefit. For example, a life insurance company sets rewards for achievement and stepwise targets so as to be an incentive actively worked on by insurance contractors. For example, a life insurance company provides planning of an insurance product based on a health improvement status of an insurance contractor. The health insurance union periodically refers to the disease risk score of the insurance contractor, and provides health support and counseling by an expert according to a change in the score. For example, the life insurance company holds regular consultation meetings and events, and provides information related to health maintenance using the measurement device 30. The life insurance company accepts opinions and demands of the insurance contractor. The health insurance union periodically verifies and evaluates whether the health improvement and the medical cost reduction of the insurance contractor have been implemented by applying the disease risk estimation system 3.

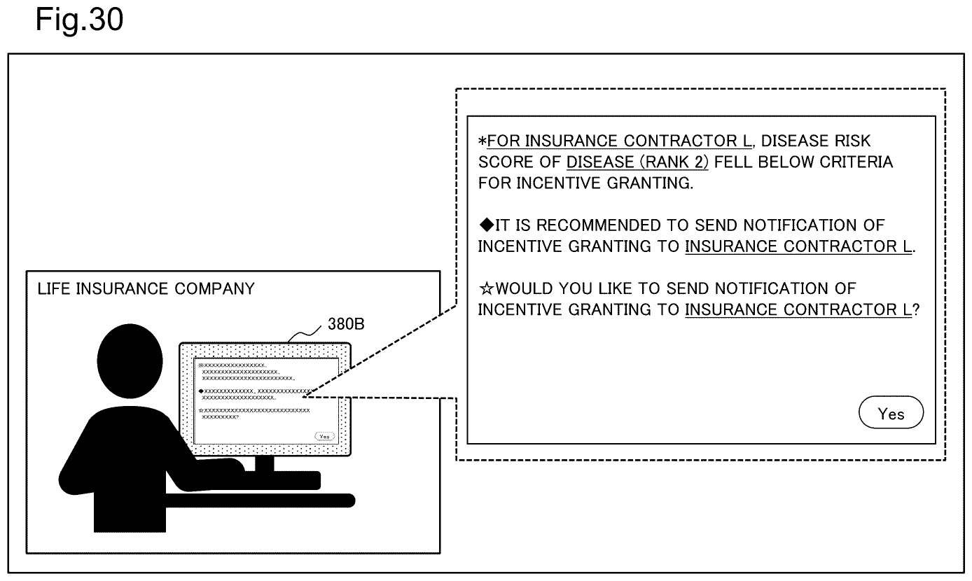

FIG. 30 illustrates an example in which the disease risk information related to the insurance contractor is displayed on the screen of a terminal device 380B used in the health insurance union. On the screen of the terminal device 380B, information relevant to the disease risk score of “For the insurance contractor L, the disease risk score of the disease (rank 2) fell below the criteria for incentive granting.” is displayed. Proposal information “It is recommended to send notification of incentive granting to the insurance contractor L.” is displayed on the screen of the terminal device 380B. This proposal information is displayed at an appropriate timing for granting an incentive from the life insurance company to the insurance contractor. Further, proposal information “Would you like to send a notification of incentive granting transmitted to the insurance contractor L?” is displayed on the screen of the terminal device 380B. A clerical staff who has confirmed the disease risk information displayed on the screen of the terminal device 380B can notify the insurance contractor of incentive granting. For example, when a “YES” button displayed on the screen of the terminal device 380B is pressed, a notification of incentive granting is transmitted to the mobile terminal carried by the insurance contractor.

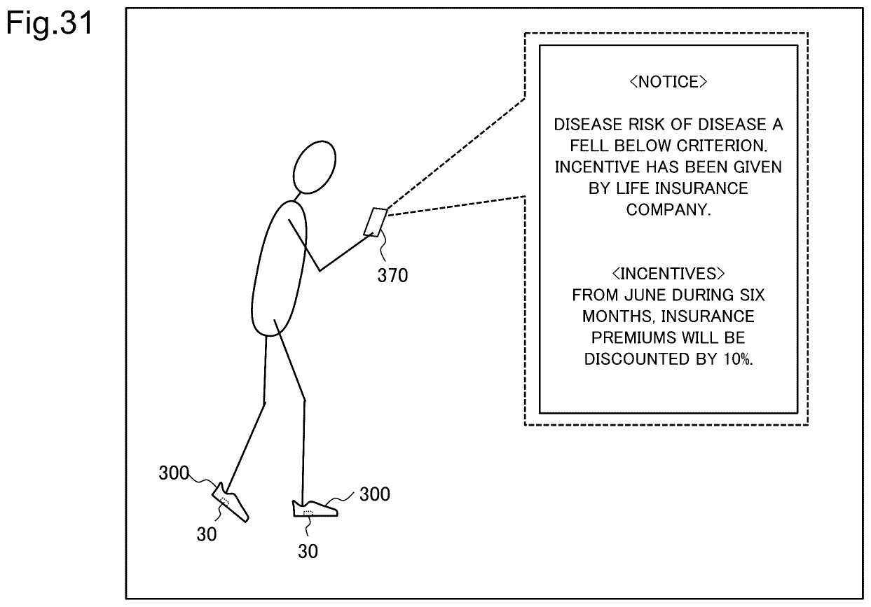

FIG. 31 illustrates an example in which the information relevant to the notification of incentive granting transmitted from the terminal device 380B of the life insurance company is displayed on the screen of the mobile terminal 370 carried by the insurance contractor walking in the shoe 300 on which the measurement device 30 is disposed. In the example of FIG. 31, a notification “The disease risk of disease A fell below the criterion. An incentive has been given by a life insurance company.” is displayed on the screen of the mobile terminal 370. In the example of FIG. 31, information related to incentive granting such as “From June during six months, the insurance premiums will be discounted by 10%.” is displayed on the screen of the mobile terminal 370 according to the disease risk score.

The insurance contractor who has confirmed the information related to the incentive granting displayed on the display unit of the mobile terminal 370 can recognize that the incentive is received. The notification of incentive granting may be provided to a person other than the insurance contractor. For example, the notification of incentive granting may be transmitted to a terminal device (not illustrated) to be used, such as a doctor or a trainer who manages the physical condition of the insurance contractor, a family of the insurance contractor, or a boss of a company of the insurance contractor. For example, the notification of incentive granting may be recorded in a database (not illustrated) constructed for the purpose of health management or the like. The transmission destination and use of the notification of incentive granting are not particularly limited.

As described above, the disease risk estimation system according to the present example embodiment includes the measurement device and the disease risk estimation device. The measurement device is installed on the footwear of the subject as an estimation target of the disease risk information. The measurement device measures spatial acceleration and spatial angular velocity. The measurement device generates sensor data using the measured spatial acceleration and spatial angular velocity. The measurement device transmits the generated sensor data to the disease risk estimation device. The disease risk estimation device includes an acquisition unit, a risk estimation unit, a proposal information generation unit, and an output unit. The acquisition unit acquires sensor data measured in accordance with the movement of the foot of the subject who is an estimation target of the disease risk. The risk estimation unit includes a calculation unit and an estimation unit. The calculation unit calculates a gait index using the sensor data. The estimation unit inputs data including the gait index calculated using the sensor data to the disease risk estimation model. The disease risk estimation model outputs a disease risk score indicating a degree of the disease risk related to a disease according to an input of data including a gait index. The estimation unit estimates the disease risk reflecting a risk for each disease according to the disease risk score output from the disease risk estimation model. The change tendency determination unit determines the disease risk for each disease according to the change tendency of the disease risk score. The proposal information generation unit generates proposal information for the insurance-related institution according to the disease risk reflecting a risk for each disease. The output unit outputs disease risk information relevant to the estimated disease risk.

As described above, the disease risk estimation device according to the present example embodiment estimates the disease risk reflecting a risk for each disease using the sensor data measured in accordance with the sensor data related to the movement of the foot of the subject. The disease risk estimation device according to the present example embodiment generates proposal information for an insurance-related institution according to the disease risk reflecting a risk for each disease. That is, according to the present example embodiment, it is possible to generate proposal information for an insurance-related institution using sensor data measured for the subject.

In an aspect of the present example embodiment, the insurance-related institution is a health insurance union. The acquisition unit acquires sensor data measured according to the gait of the insured person of the health insurance union. The risk estimation unit estimates the disease risk reflecting a risk for each disease using the acquired sensor data. The proposal information generation unit generates proposal information including a timing of notifying the insured person of the specific health guidance according to the disease risk reflecting the estimated risk for each disease. The output unit transmits proposal information including a timing at which specific health guidance is notified to a terminal device used in the health insurance union. According to the present aspect, it is possible to generate proposal information including a timing at which specific health guidance is notified to an insured person using sensor data measured for the insured person.

In an aspect of the present example embodiment, the insurance-related institution is a life insurance company. The acquisition unit acquires sensor data measured in accordance with the gait of an insurance contractor of a life insurance company. The risk estimation unit estimates the disease risk reflecting a risk for each disease using the acquired sensor data. The proposal information generation unit generates proposal information including a timing of granting an incentive to the insurance contractor according to the disease risk reflecting the estimated risk for each disease. The output unit transmits proposal information including a timing of granting an incentive to a terminal device used in a life insurance company. According to the present aspect, it is possible to generate proposal information including a timing of granting an incentive to an insurance contractor using sensor data measured for the insurance contractor.

### Fourth Example Embodiment

Next, a disease risk estimation device according to a fourth example embodiment will be described with reference to the drawings. The disease risk estimation device of the present example embodiment has a simplified configuration of the disease risk estimation device included in the disease risk estimation system of the first to third example embodiments.

### (Configuration)

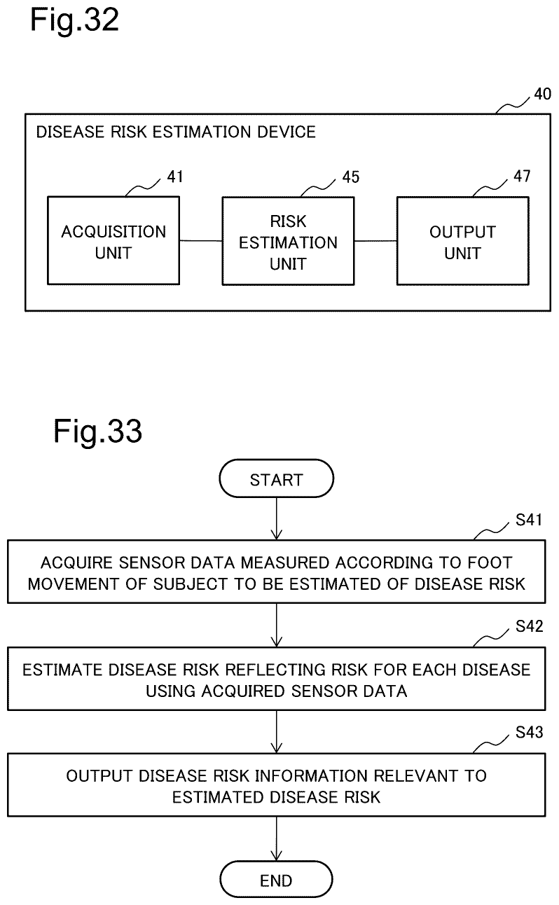

FIG. 32 is a block diagram illustrating an example of a configuration of a disease risk estimation device 40 in the present disclosure. The disease risk estimation device 40 includes an acquisition unit 41, a risk estimation unit 45, and an output unit 47.

The acquisition unit 41 acquires sensor data measured according to the movement of the foot of the subject who is an estimation target of the disease risk. The risk estimation unit 45 estimates the disease risk reflecting a risk for each disease using the acquired sensor data. The output unit 47 outputs disease risk information relevant to the estimated disease risk.

### (Operation)

Next, the operation of the disease risk estimation device 40 will be described with reference to the drawings. FIG. 33 is a flowchart for explaining an example of the operation of the disease risk estimation device 40. In the description of the processing along the flowchart of FIG. 33, the components of the disease risk estimation device 40 will be described as the operation subject. The operation subject of the processing along the flowchart of FIG. 33 may be the disease risk estimation device 40.

In FIG. 33, first, the acquisition unit 41 acquires sensor data measured according to the movement of the foot of the subject who is an estimation target of the disease risk (step S41).

The risk estimation unit 45 estimates the disease risk reflecting a risk for each disease using the acquired sensor data (step S42).

The output unit 47 outputs disease risk information relevant to the estimated disease risk (step S43).

As described above, the disease risk estimation device according to the present example embodiment estimates the disease risk reflecting a risk for each disease using the sensor data measured in accordance with the sensor data related to the movement of the foot of the subject. That is, according to the present example embodiment, the disease risk reflecting a risk for each disease can be estimated using the sensor data measured according to the movement of the foot.

### (Hardware)

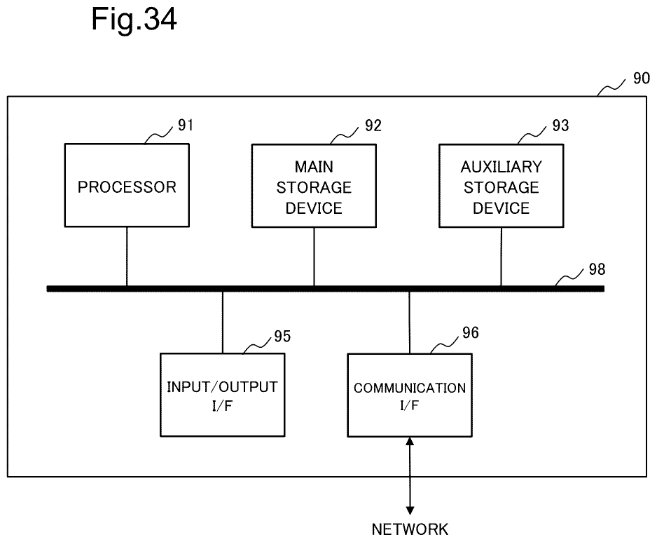

Next, a hardware configuration for executing control and processing in the present disclosure will be described with reference to the drawings. Here, an example of such a hardware configuration is an information processing device 90 (computer) in FIG. 34. The information processing device 90 of FIG. 34 is the configuration example for executing the control and processing in the present disclosure, and does not limit the scope of the present disclosure.

As illustrated in FIG. 34, the information processing device 90 includes a processor 91, a main storage device 92, an auxiliary storage device 93, an input/output interface 95, and a communication interface 96. In FIG. 34, the interface is abbreviated as an interface (I/F). The processor 91, the main storage device 92, the auxiliary storage device 93, the input/output interface 95, and the communication interface 96 are data-communicably connected to each other via a bus 98. The processor 91, the main storage device 92, the auxiliary storage device 93, and the input/output interface 95 are connected to a network such as the Internet or an intranet via the communication interface 96.

The processor 91 develops a program (instruction) stored in the auxiliary storage device 93 or the like in the main storage device 92. For example, the program is a software program for executing the control and processing in the present disclosure. The processor 91 executes the program developed in the main storage device 92. The processor 91 executes the control and processing in the present disclosure by executing the program.

The main storage device 92 is a region in which a program is developed. A program stored in the auxiliary storage device 93 or the like is developed in the main storage device 92 by the processor 91. The main storage device 92 is achieved by, for example, a volatile memory such as a dynamic random access memory (DRAM). A nonvolatile memory such as a magneto resistive random access memory (MRAM) may be configured/added as the main storage device 92.

The auxiliary storage device 93 stores various types of data such as programs. The auxiliary storage device 93 is achieved by local disks such as hard disks or flash memories. Various types of data may be stored in the main storage device 92, and the auxiliary storage device 93 may be omitted.

The input/output interface 95 is an interface for connecting the information processing device 90 and a peripheral device in accordance with a standard or a specification. The communication interface 96 is an interface for connecting to an external system or device through a network such as the Internet or an intranet in accordance with a standard or a specification. The input/output interface 95 and the communication interface 96 may be shared as an interface connected to an external device.

Input devices such as a keyboard, a mouse, and a touch panel may be connected to the information processing device 90 as necessary. These input devices are used to input information and settings. In a case where the touch panel is used as the input device, a screen having a touch panel function serves as an interface. The processor 91 and the input device are connected via the input/output interface 95.

The information processing device 90 may be provided with a display device for displaying information. In a case where the display device is provided, the information processing device 90 includes a display control device (not illustrated) for controlling display of the display device. The information processing device 90 and the display device are connected via the input/output interface 95.

The information processing device 90 may be provided with a drive device. The drive device mediates reading of data and a program stored in a recording medium and writing of a processing result of the information processing device 90 to the recording medium between the processor 91 and the recording medium (program recording medium). The information processing device 90 and the drive device are connected via the input/output interface 95.

The above is an example of the hardware configuration for enabling the control and processing in the present disclosure. The hardware configuration of FIG. 34 is an example of the hardware configuration for executing the control and processing in the present disclosure, and does not limit the scope of the present disclosure. A program for causing a computer to execute the control and processing in the present disclosure is also included in the scope of the present disclosure.

A program recording medium recording a program in the present disclosure is also included in the scope of the present disclosure. The recording medium can be achieved by, for example, an optical recording medium such as a compact disk (CD) or a digital versatile disk (DVD). The recording medium may be achieved by a semiconductor recording medium such as a universal serial bus (USB) memory or a secure digital (SD) card. The recording medium may be achieved by a magnetic recording medium such as a flexible disk, or other recording media. When a program executed by the processor is recorded in a recording medium, the recording medium is relevant to a program recording medium.

The components in the present disclosure may be combined in any manner. The components in the present disclosure may be achieved by software. The components in the present disclosure may be achieved by a circuit.

While the present disclosure has been particularly shown and described with reference to example embodiments thereof, the present disclosure is not limited to these example embodiments. It will be understood by those of ordinary skill in the art that various changes in form and details may be made therein without departing from the spirit and scope of the present disclosure as defined by the claims. And each embodiment can be appropriately combined with other embodiments.

Some or all of the above example embodiments may also be described as the following Supplementary Notes, but are not limited to the following.

### (Supplementary Note 1)

A disease risk estimation device including:
an acquisition unit that acquires sensor data measured in accordance with a movement of a foot of a subject who is an estimation target of a disease risk;
a risk estimation unit that estimates a disease risk reflecting a risk for each disease using the acquired sensor data; and
an output unit that outputs disease risk information relevant to the estimated disease risk.

### (Supplementary Note 2)

The disease risk estimation device according to Supplementary Note 1, in which
the risk estimation unit includes:
a calculation unit that calculates a gait index using the sensor data; and
an estimation unit that inputs data including the gait index calculated using the sensor data to a disease risk estimation model that outputs a disease risk score indicating a degree of the disease risk related to the disease according to an input of the data including the gait index, and estimates disease risk information relevant to the disease risk score output from the disease risk estimation model.

### (Supplementary Note 3)

The disease risk estimation device according to Supplementary Note 2, in which
the estimation unit is configured to execute:
calculating the disease risk score reflecting a risk for each disease by multiplying the disease risk score by a weight for each disease according to a rank indicating a risk of a disease.

### (Supplementary Note 4)

The disease risk estimation device according to Supplementary Note 2, in which
the estimation unit is configured to execute:
calculating the disease risk score reflecting a risk for each combination of diseases by multiplying the disease risk score by a weight for the each combination of diseases.

### (Supplementary Note 5)

The disease risk estimation device according to Supplementary Note 2, including a change tendency determination unit that determines a disease risk for each disease according to a change tendency of the disease risk score.

### (Supplementary Note 6)

The disease risk estimation device according to Supplementary Note 5, in which
the change tendency determination unit is configured to execute:
determining that a disease risk is high with respect to a disease for which the disease risk score is on an increasing tendency; and
determining that the disease risk is low with respect to a disease on any one of a decreasing tendency and a stagnation tendency of the disease risk score.

### (Supplementary Note 7)

The disease risk estimation device according to Supplementary Note 5, in which
the change tendency determination unit is configured to execute:
determining that a disease risk is high with respect to a disease for which the disease risk score exceeds a threshold; and
determining that a disease risk is low with respect to a disease for which the disease risk score is below the threshold.

### (Supplementary Note 8)

The disease risk estimation device according to Supplementary Note 2, including a proposal information generation unit that generates proposal information for an insurance-related institution according to the disease risk reflecting a risk for each disease.

### (Supplementary Note 9)

The disease risk estimation device according to Supplementary Note 8, in which
the insurance-related institution is a health insurance union,
the acquisition unit is configured to execute
acquiring the sensor data measured according to a gait of an insured person of the health insurance union,
the risk estimation unit is configured to execute
estimating the disease risk reflecting a risk for each disease using the acquired sensor data,
the proposal information generation unit is configured to execute
generating the proposal information including a timing of notifying the insured person of a specific health guidance according to the disease risk reflecting the estimated risk for each disease, and
the output unit is configured to execute
transmitting, to a terminal device used in the health insurance union, the proposal information including a timing at which the specific health guidance is notified.

### (Supplementary Note 10)

The disease risk estimation device according to Supplementary Note 8, in which
the insurance-related institution is a life insurance company,
the acquisition unit is configured to execute
acquiring the sensor data measured according to a gait of an insurance contractor of the life insurance company,
the risk estimation unit is configured to execute
estimating the disease risk reflecting a risk for each disease using the acquired sensor data,
the proposal information generation unit is configured to execute
generating the proposal information including a timing of granting an incentive to the insurance contractor according to the disease risk reflecting the estimated risk for each disease, and
the output unit is configured to execute
transmitting the proposal information including the timing of granting the incentive to a terminal device used in the life insurance company.

### (Supplementary Note 11)

The disease risk estimation device according to Supplementary Note 2, in which
the disease risk estimation model is
a model learned using a machine learning method, and includes an incomplete heterogeneous variational autoencoder.

### (Supplementary Note 12)

A disease risk estimation system including:
the disease risk estimation device according to any one of Supplementary Note 1 to 11; and
a measurement device that is installed on footwear of the subject who is an estimation target of the disease risk information, measures spatial acceleration and spatial angular velocity, generates the sensor data using the measured spatial acceleration and spatial angular velocity, and transmits the generated sensor data to the disease risk estimation device.

### (Supplementary Note 13)

The disease risk estimation system according to Supplementary Note 12, in which
the disease risk estimation device is configured to execute:
displaying the disease risk information optimized for the subject on a screen of a terminal device browsable by a user.

### (Supplementary Note 14)

A disease risk estimation method for causing a computer to execute:
acquiring sensor data measured according to a movement of a foot of a subject who is an estimation target of a disease risk;
estimating a disease risk reflecting a risk for each disease using the acquired sensor data; and
outputting disease risk information relevant to the estimated disease risk.

### (Supplementary Note 15)

The disease risk estimation method according to Supplementary Note 14, in which
the computer executes:
calculating a gait index using the sensor data;
inputting data including the gait index calculated using the sensor data to a disease risk estimation model that outputs a disease risk score indicating a degree of a disease risk related to the disease according to an input of the data including the gait index; and
estimating disease risk information according to the disease risk score output from the disease risk estimation model.

### (Supplementary Note 16)

The disease risk estimation method according to Supplementary Note 15, in which
the computer executes:
calculating the disease risk score reflecting a risk for each disease by multiplying the disease risk score by a weight for each disease according to a rank indicating a risk of a disease.

### (Supplementary Note 17)

The disease risk estimation method according to Supplementary Note 15, in which
the computer executes:
calculating the disease risk score reflecting a risk for each combination of diseases by multiplying the disease risk score by a weight for the each combination of diseases.

### (Supplementary Note 18)

The disease risk estimation method according to Supplementary Note 15, in which
the computer executes:
determining a disease risk for each disease according to a change tendency of the disease risk score.

### (Supplementary Note 19)

The disease risk estimation method according to Supplementary Note 15, in which
the computer executes:
generating proposal information for an insurance-related institution according to the disease risk reflecting a risk for each disease.

### (Supplementary Note 20)

A computer-readable non-transitory recording medium having recorded therein a program for causing a computer to execute:
a process of acquiring sensor data measured in accordance with a movement of a foot of a subject who is an estimation target of a disease risk;
a process of estimating a disease risk reflecting a risk for each disease using the acquired sensor data; and
a process of outputting disease risk information relevant to the estimated disease risk.

### REFERENCE SIGNS LIST

1, 2, 3 disease risk estimation system
10, 20, 30 measurement device
13, 23, 33, 40 disease risk estimation device
15 risk estimation unit
41 acquisition unit
45 risk estimation unit
47 output unit
110 sensor
111 acceleration sensor
112 angular velocity sensor
113 control unit
115 communication unit
117 power supply
130, 230, 330 calculation unit
131, 231, 331 acquisition unit
132 waveform processing unit
133 gait index calculation unit
134, 234, 334 storage unit
135 physical ability estimation unit
136 disease risk estimation unit
137, 237, 337 output unit
140, 240 estimation unit
245 change tendency determination unit
345 proposal information generation unit

What is claimed is:

1. A disease risk estimation device comprising:
a memory storing instructions; and
a processor connected to the memory and configured to execute the instructions to:
acquire sensor data measured in accordance with a movement of a foot of a subject who is an estimation target of a disease risk;
estimate a disease risk reflecting a risk for each disease using the acquired sensor data; and
output disease risk information relevant to the estimated disease risk.

2. The disease risk estimation device according to claim 1, wherein
the processor is configured to execute the instructions to:
calculate a gait index using the sensor data; and
input data including the gait index calculated using the sensor data to a disease risk estimation model that outputs a disease risk score indicating a degree of the disease risk related to the disease according to an input of data including the gait index, and estimates disease risk information relevant to the disease risk score output from the disease risk estimation model.

3. The disease risk estimation device according to claim 2, wherein
the processor execute is configured to execute the instructions to:
calculate the disease risk score reflecting a risk for each disease by multiplying the disease risk score by a weight for each disease according to a rank indicating a risk of a disease.

4. The disease risk estimation device according to claim 2, wherein
the processor execute is configured to execute the instructions to:
calculate the disease risk score reflecting a risk for each combination of diseases by multiplying the disease risk score by a weight for the each combination of diseases.

5. The disease risk estimation device according to claim 2, wherein
the processor is configured to execute the instructions to
determine a disease risk for each disease according to a change tendency of the disease risk score.

6. The disease risk estimation device according to claim 5, wherein
the processor is configured to execute the instructions to:
determine that a disease risk is high with respect to a disease for which the disease risk score is on an increasing tendency; and
determine that the disease risk is low with respect to a disease on any one of a decreasing tendency and a stagnation tendency of the disease risk score.

7. The disease risk estimation device according to claim 5, wherein
the processor is configured to execute the instructions to:
determine that a disease risk is high with respect to a disease for which the disease risk score exceeds a threshold; and
determine that a disease risk is low with respect to a disease for which the disease risk score is below the threshold.

8. The disease risk estimation device according to claim 2, wherein
the processor is configured to execute the instructions to
generate proposal information for an insurance-related institution according to the disease risk reflecting a risk for each disease.

9. The disease risk estimation device according to claim 8, wherein
the insurance-related institution is a health insurance union, and
the processor is configured to execute the instructions to:
acquire the sensor data measured according to a gait of an insured person of the health insurance union,
estimate the disease risk reflecting a risk for each disease using the acquired sensor data,
generate the proposal information including a timing of notifying the insured person of a specific health guidance according to the disease risk reflecting the estimated risk for each disease, and
transmit, to a terminal device used in the health insurance union, the proposal information including a timing at which the specific health guidance is notified.

10. The disease risk estimation device according to claim 8, wherein
the insurance-related institution is a life insurance company,
the processor is configured to execute the instructions to;
acquire the sensor data measured according to a gait of an insurance contractor of the life insurance company,
estimate the disease risk reflecting a risk for each disease using the acquired sensor data,
generate the proposal information including a timing of granting an incentive to the insurance contractor according to the disease risk reflecting the estimated risk for each disease, and
transmit the proposal information including the timing of granting the incentive to a terminal device used in the life insurance company.

11. The disease risk estimation device according to claim 2, wherein
the disease risk estimation model is
a model learned using a machine learning method, and includes an incomplete heterogeneous variational autoencoder.

12. A disease risk estimation system comprising:
the disease risk estimation device according to claim 1; and
a measurement device that is installed on footwear of the subject who is an estimation target of the disease risk information, wherein
the measurement device includes
a sensor that measures spatial acceleration and spatial angular velocity,
a controller that generates the sensor data using the measured spatial acceleration and spatial angular velocity, and
a transmitter that transmits the generated sensor data to the disease risk estimation device via wireless communication.

13. The disease risk estimation system according to claim 12, wherein
the processor of the disease risk estimation device is configured to execute the instructions to:
display the disease risk information optimized for the subject on a screen of a terminal device browsable by a user.

14. A disease risk estimation method for causing a computer to execute:
acquiring sensor data measured according to a movement of a foot of a subject who is an estimation target of a disease risk;
estimating a disease risk reflecting a risk for each disease using the acquired sensor data; and
outputting disease risk information relevant to the estimated disease risk.

15. The disease risk estimation method according to claim 14, wherein
the computer executes:
calculating a gait index using the sensor data;
inputting data including the gait index calculated using the sensor data to a disease risk estimation model that outputs a disease risk score indicating a degree of a disease risk related to the disease according to an input of the data including the gait index; and
estimating disease risk information according to the disease risk score output from the disease risk estimation model.

16. The disease risk estimation method according to claim 15, wherein
the computer executes:
calculating the disease risk score reflecting a risk for each disease by multiplying the disease risk score by a weight for each disease according to a rank indicating a risk of a disease.

17. The disease risk estimation method according to claim 15, wherein
the computer executes:
calculating the disease risk score reflecting a risk for each combination of diseases by multiplying the disease risk score by a weight for the each combination of diseases.

18. The disease risk estimation method according to claim 15, wherein
the computer executes:
determining a disease risk for each disease according to a change tendency of the disease risk score.

19. The disease risk estimation method according to claim 15, wherein
the computer executes:
generating proposal information for an insurance-related institution according to the disease risk reflecting a risk for each disease.

20. A computer-readable non-transitory recording medium having recorded therein a program for causing a computer to execute:
a process of acquiring sensor data measured in accordance with a movement of a foot of a subject who is an estimation target of a disease risk;
a process of estimating a disease risk reflecting a risk for each disease using the acquired sensor data; and
a process of outputting disease risk information relevant to the estimated disease risk.
